(19) United States
(12) Patent Application Publication
(10) Pub. No.: US 2026/0196301 A1
(43) Pub. Date: Jul. 9, 2026

(54) IDENTIFYING CELL-TYPES

(71) Applicant: Sanofi, PARIS (FR)

(72) Inventors: Giorgio Gaglia, Cambridge, MA (US); Nima Nouri, Burlington, MA (US); Virginia Savova, Brookline, MA (US)

(21) Appl. No.: 19/132,632

(22) PCT Filed: Nov. 27, 2023
(86) PCT No.: PCT/US2023/081169

§ 371 Date: May 23, 2025

Related U.S. Application Data
(60) Provisional application No. 63/428,267, filed on Nov. 28, 2022.

(30) Foreign Application Priority Data
Mar. 10, 2023 (EP) 23315048.1

(51) Int. Cl. G16B 30/00; G16B 25/10

(52) U.S. Cl. CPC G16B 30/00; G16B 25/10

(57) ABSTRACT

Provided herein are compositions and methods for identifying the cell-type origin of cells based on RNA-sequencing (RNA-seq) data. The methods provided herein can include steps of receiving a plurality of sequencing read counts; providing a set of cell types to be detected and an associated gene set G for each cell type, such that each gene set G comprises a plurality of genes g; scoring the sequencing read counts to generate an assignment score; and assigning each cell of the plurality of cells to a cell-type with the highest assignment score to identify the cell-type origin of each cell. The methods can be implemented using, for example, a computer system.

### CROSS-REFERENCE TO RELATED APPLICATIONS

This application claims the benefit of U.S. Provisional Patent Application No. 63/428,267, filed Nov. 28, 2022, and European Patent Application No. 23315048.1, filed Mar. 10, 2023, the contents of which are incorporated herein by reference in their entirety.

### TECHNICAL FIELD

This disclosure relates to identifying cell-types based on RNA-sequencing (RNA-seq) data.

### BACKGROUND

Single-cell RNA sequencing (scRNA-seq) experiments provide opportunities to peer into complex tissues at single-cell resolution. However, insightful biological interpretation of scRNA-seq data relies upon precise identification of cell types. The ability to identify the origin of a cell quickly and accurately from scRNA-seq data can improve downstream analyses. However, identifying cell types can be challenging due to phenotypic and cell-state variability.

Manual annotation using standard scRNA-seq data analysis workflows typically rely on identifying cell types through canonical marker genes by manually inspecting the expression of cell type-specific markers, based on which each group is assigned to a specific cell type. These marker features are either previously known from prior research or they are identified using differential expression analysis of the given cell group against the rest of a dataset. However, this strategy is time-consuming, biased, and prone to error. Manual annotation requires the high-dimensional data to be pre-processed for user visualization. This preprocessing reshapes the data so that cells with similar biological patterns of transcripts end up with similar transformed measurements, and hence fall closer to each other in the reduce-dimension gene-expression space. These preprocessing steps result in unintended distortions that affect downstream analysis. In addition, clustering algorithms reduce the variation present in high-dimensional input, hence quenching the biological signal. Lastly, the manual cell-type assignment is user-dependent and susceptible to investigator bias.

### SUMMARY

The present disclosure is based, in part, on development of a cell-type annotation method that operates at individual cell resolution by applying a scoring system to scRNA-seq data based on sets of marker genes associated with cell types. The methods disclosed herein are transformation- and cluster-free which avoid undesirable distortions caused by preprocessing steps. Disclosed herein are the basis of the method, in silico experiments benchmarking the method's performance on simulated data, and a comparison of automatic annotations from the method with manually annotated experimental data from the Tabula Sapiens Consortium (Tabula Sapiens Consortium. The Tabula Sapiens: A multiple-organ, single-cell transcriptomic atlas of humans. Science. 2022 May 13;376(6594).).

According to one aspect of the present disclosure, methods for identifying a cell-type origin of each cell of a plurality of cells is provided. In one aspect, the methods can include receiving a plurality of sequencing read counts, providing a set of cell types to be detected and an associated gene set G for each cell type, such that each gene set G includes a plurality of genes g, scoring the sequencing read counts to generate an assignment score, and assigning each cell of the plurality of cells to a cell-type with the highest assignment score to identify the cell-type origin of each cell. In some embodiments, the methods further include trimming the sequencing read counts to remove ambiguous calls. In some embodiments, the methods further include smoothing annotations of the sequencing read counts. In some embodiments, the plurality of sequencing read counts are generated by RNA sequencing (RNA-seq). In some embodiments, the plurality of sequencing read counts are generated by single cell RNA sequencing (scRNA-seq) of each cell of the plurality of cells. In some embodiments, the scoring step includes sorting the plurality of genes g of the associated gene set G exhibiting non-zero expression from high to low expression. In some embodiments, the scoring step further includes generating, from the sorted genes of the associated gene set, a ranked vector of length N. In some embodiments, the scoring step further includes converting the ranked vector of length N to a binary sequence s, such that genes g that are included in a specific gene-set G are substituted by 1 or 0.

In some embodiments, the binary sequence s is represented by the following formula:

$$s = {\{ \begin{matrix}1 : {g}_{n} ∈ G \\ 0 : {g}_{n} ∉ G\end{matrix} \}}_{1 \leq n \leq N} .$$

In some embodiments, the scoring step further includes performing a cumulative sum over the binary sequence s up to each element k of the binary vector. In some embodiments, the scoring step further includes performing a sum over all generated sequential 1-to-k partial sums to produce an assignment-score S for a given cell according to the following formula:

$$S = {\sum }_{k = 1}^{N} {\sum }_{n = 1}^{k} {s}_{n} .$$

In some embodiments, the scoring step further includes performing one or more of the preceding steps for each cell of the plurality of cells and gene sets for a total of M gene sets. In some embodiments, the scoring step further includes transforming an input gene-by-cell expression matrix into a gene-set-by-cell assignment-score matrix, wherein the input gene-by-cell expression matrix comprises [number of genes×number of cells] and the gene-set-by-cell assignment-score matrix comprises [number of gene sets×number of cells].

In some embodiments, each cells of the plurality of cells is assigned to a cell-type representing the highest assignment score represented by the following formula:

$$max {\{ {S}_{m} \}}_{1 \leq m \leq M} .$$

In some embodiments, the trimming step further includes calculating a Gini index value for each cell of the plurality of cells. In some embodiments, the trimming step further includes transforming the gene-set-by-cell assignment-score matrix into a distribution of indexes according to the following formula:

∈[0,1]

In some embodiments, the trimming step further includes excluding cells with an index that is an outlier and/or is less than 0.5. In some embodiments, the smoothing step further includes applying a k-nearest neighbor (kNN) algorithm to the sequencing read counts.

In some embodiments, the smoothing step further includes identifying cells with a minimum of k nearest-neighbors, and if more than 50% of neighbors reach a consensus annotation, annotating the neighboring cell according to the neighbor's consensus. In some embodiments, the methods further include providing a set of negative markers, for which cells that do not express one or more negative markers are rewarded and cells that express one or more negative markers are penalized.

In some embodiments, the methods do not include normalization of the plurality of sequencing read counts. In some embodiments, the methods do not include transformation of the plurality of sequencing read counts. In some embodiments, the methods do not comprise clustering of the plurality of sequencing read counts.

In some embodiments, the plurality of cells are human cells. In some embodiments, the set of cell types to be detected include 1, 2, 3, 4, 5, 6, 7, 8, 9, or 10 or more cell types to be detected. In some embodiments, the plurality of genes g includes 10, 20, 30, 40, 50, 60, 70, 80, 90, or 100 or more genes.

In another aspect of the present disclosure, a computer program product tangibly embodied on a computer-readable medium is provided. In one aspect of the disclosure, the computer program can include instructions, that when executed by one or more processors, are configured to receive a plurality of sequencing read counts, receive a set of cell types to be detected and an associated gene set G for each cell type, such that each gene set G comprises a plurality of genes g, score the sequencing read counts to generate an assignment score, and assign each cell of the plurality of cells to a cell-type with the highest assignment score to identify the tissue-type origin of each cell. In some embodiments, the computer program further includes instructions that, when executed by the one or more processors, are configured to trim the sequencing read counts to remove ambiguous calls. In some embodiments, the computer program further includes instructions that, when executed by the one or more processors, are configured to smooth annotations of the sequencing read counts.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the methods and compositions will be apparent from the following detailed description and figures, and from the claims.

### DESCRIPTION OF DRAWINGS

FIG. 1 is a flowchart describing sequential steps of the methods disclosed herein.

FIG. 2 is a flowchart further describing sequential steps of the methods disclosed herein.

FIG. 3A is a schematized representation of an input of the method disclosed herein, wherein expression of single genes (rows) are evaluated for each single cell (columns) in an experiment, based on scRNA-seq data.

FIG. 3B is a schematized representation of a scoring step of the method disclosed herein, wherein single cells (columns) are scored relative to provided gene sets (rows).

FIG. 3C is a schematized representation of a trimming step of the method disclosed herein, wherein the Gini index is used as a measure of dispersion to identify ambiguous calls and unidentified unclassified cells for the remaining steps of the method.

FIG. 3D is a schematized representation of a smoothing step of the method disclosed herein, wherein cells with a minimum of k nearest-neighbors are identified according to aggregating information from cells with similar genome-wide expression profiles (neighbors).

FIG. 4A is a table showing the numbers of cells, genes, and cell-types of simulated data used to benchmark the cell-type identification methods disclosed herein.

FIG. 4B is a diagram depicting study design for benchmarking the cell-type identification methods disclosed herein using simulated data, including a training dataset, marker gene-sets, and test datasets.

FIG. 4C is a series of three plots, showing benchmarking performance of the cell-type identification methods disclosed herein using simulated data. Sensitivity (left), specificity (center), and F1-score (right) are shown.

FIG. 4D is a series of three plots, showing benchmarking performance of the cell-type identification methods disclosed herein using simulated data for discovering novel cell-types, with simulated data corresponding to 5,000 cells, 10,000 cells, and 15,000 cells. Sensitivity (left), specificity (center), and F1-score (right) are shown.

FIG. 5 is a diagram of computer system components that can be used to implement methods for identifying a cell-type origin of each cell of a plurality of cells based on scRNA-seq data.

FIG. 6A is a cluster plot depicting a two-dimensional map organizing cells on the basis of the similarity of their gene expression profiles. Data from the Tabula Sapiens Consortium (left) are compared to annotations using the cell-type identification method disclosed herein (right) for peripheral blood mononuclear cells (PBMCs).

FIG. 6B is a plot of the Jaccard similarity index comparing data from the Tabula Sapiens Consortium to annotations using the cell-type identification method disclosed herein for peripheral blood mononuclear cells (PBMCs).

FIG. 6C is a plot of expression of PBMC marker genes plotted by cell type (y-axis) and canonical marker (x-axis) comparing data from the Tabula Sapiens Consortium to annotations using the cell-type identification method disclosed herein. Circle size corresponds to percentage of cells expressing the marker, and shading intensity corresponds to expression level.

FIG. 7A is a series of t-stochastic neighbor embedding (t-SNE) plots for each of nine canonical markers for T cells (TC) and natural killer cells (NK).

FIG. 7B is a cluster plot depicting a two-dimensional map organizing cells on the basis of the similarity of their gene expression profiles. Data from the Tabula Sapiens Consortium (top) are compared to annotations using the cell-type identification method disclosed herein (bottom) for T cells (TC) and natural killer cells (NK).

FIG. 7C is a plot of expression of natural killer cell marker genes plotted by cell type (y-axis) and canonical marker (x-axis) comparing data from the Tabula Sapiens Consortium to annotations using the cell-type identification method disclosed herein. Circle size corresponds to percentage of cells expressing the marker, and shading intensity corresponds to expression level.

FIG. 7D is a plot of the Jaccard similarity index comparing data from the Tabula Sapiens Consortium to annotations using the cell-type identification method disclosed herein for T cells (TC) and natural killer cells (NK).

FIG. 7E is a plot of expression level of canonical T cell (TC) and natural killer (NK) cell markers for a population of cells that were identified by the cell-type identification method disclosed herein as NK cells and by the Tabula study as T cells.

FIG. 8A is a series of t-stochastic neighbor embedding (t-SNE) plots for each of four canonical markers for T cells (TC).

FIG. 8B is a cluster plot depicting a two-dimensional map organizing cells on the basis of the similarity of their gene expression profiles. Data from the Tabula Sapiens Consortium (top) are compared to annotations using the cell-type identification method disclosed herein (bottom) for T cells (TC).

FIG. 8C is a plot of expression of T cell marker genes plotted by cell type (y-axis) and canonical marker (x-axis) comparing data from the Tabula Sapiens Consortium to annotations using the cell-type identification method disclosed herein. Circle size corresponds to percentage of cells expressing the marker, and shading intensity corresponds to expression level.

FIG. 8D is a plot of the Jaccard similarity index comparing data from the Tabula Sapiens Consortium to annotations using the cell-type identification method disclosed herein for T cells (TC).

FIG. 8E is a plot of expression levels of three T-cell canonical markers, CD4, CD8A, and CD8B, for a mixed population of cells that were identified as CD4⁺ by the cell-type identification method disclosed herein and as CD8⁺ by the Tabula study (left), or as CD8⁺ by the cell-type identification method disclosed herein and as CD4⁺ by the Tabula study (right).

FIG. 9A is a cluster plot depicting a two-dimensional map organizing cells on the basis of the similarity of their gene expression profiles. Data from the Tabula Sapiens Consortium (left) are compared to annotations using the cell-type identification method disclosed herein (right) for macrophages (Mac) and monocytes (Mon).

FIG. 9B is a plot of expression of macrophage and monocyte canonical markers plotted by cell type (y-axis) and canonical marker (x-axis) comparing data from the Tabula Sapiens Consortium to annotations using the cell-type identification method disclosed herein. Circle size corresponds to percentage of cells expressing the marker, and shading intensity corresponds to expression level.

FIG. 9C is a plot of the Jaccard similarity index comparing data from the Tabula Sapiens Consortium to annotations using the cell-type identification method disclosed herein for macrophages (Mac) and monocytes (Mon).

FIG. 9D is a series of heatmaps of CD16 and CD14 expression for classical, intermediate, and non-classical monocytes as annotated by the cell-type identification method disclosed herein (top panels) and by the Tabula study (bottom panels).

FIG. 10A is a cluster plot depicting a two-dimensional map organizing cells on the basis of the similarity of their gene expression profiles. Data from the Tabula Sapiens Consortium (left) are compared to annotations using the cell-type identification method disclosed herein (right) for naïve B cells, memory B cells, and plasma cells.

FIG. 10B is a plot of expression of naïve B cell (B.Naive), memory B cell (B.Mem), and plasma cell canonical markers plotted by cell type (y-axis) and canonical marker (x-axis) comparing data from the Tabula Sapiens Consortium to annotations using the cell-type identification method disclosed herein. Circle size corresponds to percentage of cells expressing the marker, and shading intensity corresponds to expression level.

FIG. 10C is a plot of the Jaccard similarity index comparing data from the Tabula Sapiens Consortium to annotations using the cell-type identification method disclosed herein for naïve B cells (B.Naive), memory B cells (B.Mem), and plasma cells.

FIG. 10D is a plot of expression levels of B cell canonical markers for a mixed population of cells that were identified as naïve B cells by the cell-type identification method disclosed herein and as memory B cells by the Tabula study.

FIG. 11A is a cluster plot depicting a two-dimensional map organizing cells on the basis of the similarity of their gene expression profiles. Data from the Tabula Sapiens Consortium (left) are compared to annotations using the cell-type identification method disclosed herein (right) for heart tissue, including cardiac fibroblasts (CFs), cardiac muscle cells (CMC), endothelial cells (Endo), hepatocytes (Hepa), macrophages (Mac), and smooth muscle cells (SMC).

FIG. 11B is a plot of expression of cardiac fibroblasts (CFs), cardiac muscle cells (CMC), endothelial cells (Endo), hepatocytes (Hepa), macrophages (Mac), and smooth muscle cells (SMC) canonical markers plotted by cell type (y-axis) and canonical marker (x-axis) comparing data from the Tabula Sapiens Consortium to annotations using the cell-type identification method disclosed herein. Circle size corresponds to percentage of cells expressing the marker, and shading intensity corresponds to expression level.

FIG. 11C is a plot of the Jaccard similarity index comparing data from the Tabula Sapiens Consortium to annotations using the cell-type identification method disclosed herein for cardiac fibroblasts (CFs), cardiac muscle cells (CMC), endothelial cells (Endo), hepatocytes (Hepa), macrophages (Mac), and smooth muscle cells (SMC).

FIG. 12A is a cluster plot depicting a two-dimensional map organizing cells on the basis of the similarity of their gene expression profiles. Data from the Tabula Sapiens Consortium (left) are compared to annotations using the cell-type identification method disclosed herein (right) for human kidney tissue, including B cells (B), endothelial cells (Endo), Epithelial cells (Epit), macrophages (Mac), and T and natural killer cell population (TNKs).

FIG. 12B is a plot of expression of human kidney tissue canonical markers, including B cells (B), endothelial cells (Endo), Epithelial cells (Epit), macrophages (Mac), and T and natural killer cell population (TNKs) canonical markers plotted by cell type (y-axis) and canonical marker (x-axis) comparing data from the Tabula Sapiens Consortium to annotations using the cell-type identification method disclosed herein. Circle size corresponds to percentage of cells expressing the marker, and shading intensity corresponds to expression level.

FIG. 12C is a plot of the Jaccard similarity index comparing data from the Tabula Sapiens Consortium to annotations using the cell-type identification method disclosed herein for human kidney tissue cell types, including B cells (B), endothelial cells (Endo), Epithelial cells (Epit), macrophages (Mac), and T and natural killer cell population (TNKs).

FIG. 13A is a cluster plot depicting a two-dimensional map organizing cells on the basis of the similarity of their gene expression profiles. Data from the Tabula Sapiens Consortium (left) are compared to annotations using the cell-type identification method disclosed herein (right) for human lung tissue, including adventitial cells (AC), alveolar type 1 and type 2 cells (ATC), basal cells (Basal), basophils (Baso), B and Plasma cells (BPC), club cells (CC), endothelial (Endo), fibroblasts (Fibro), goblet, serous, and mucous cells (GSM), lung ciliated cells (LCC), mesothelial cells (MC), monocytes, dendritic cells, and macrophages (MPh), neutrophils (Neut), pericyte cells (PC), pulmonary ionocytes (PI), smooth muscle cells (SMC), T cells and natural killer cells (TNK).

FIG. 13B is a plot of expression of human lung tissue canonical markers, including adventitial cells (AC), alveolar type 1 and type 2 cells (ATC), basal cells (Basal), basophils (Baso), B and Plasma cells (BPC), club cells (CC), endothelial (Endo), fibroblasts (Fibro), goblet, serous, and mucous cells (GSM), lung ciliated cells (LCC), mesothelial cells (MC), monocytes, dendritic cells, and macrophages (MPh), neutrophils (Neut), pericyte cells (PC), pulmonary ionocytes (PI), smooth muscle cells (SMC), T and natural killer cells (TNK) canonical markers plotted by cell type (y-axis) and canonical marker (x-axis) comparing data from the Tabula Sapiens Consortium to annotations using the cell-type identification method disclosed herein. Circle size corresponds to percentage of cells expressing the marker, and shading intensity corresponds to expression level.

FIG. 13C is a plot of the Jaccard similarity index comparing data from the Tabula Sapiens Consortium to annotations using the cell-type identification method disclosed herein for complex human lung tissue, including adventitial cells (AC), alveolar type 1 and type 2 cells (ATC), basal cells (Basal), basophils (Baso), B and Plasma cells (BPC), club cells (CC), endothelial (Endo), fibroblasts (Fibro), goblet, serous, and mucous cells (GSM), lung ciliated cells (LCC), mesothelial cells (MC), monocytes, dendritic cells, and macrophages (MPh), neutrophils (Neut), pericyte cells (PC), pulmonary ionocytes (PI), smooth muscle cells (SMC), T and natural killer cells (TNK).

### DETAILED DESCRIPTION

### Scoring

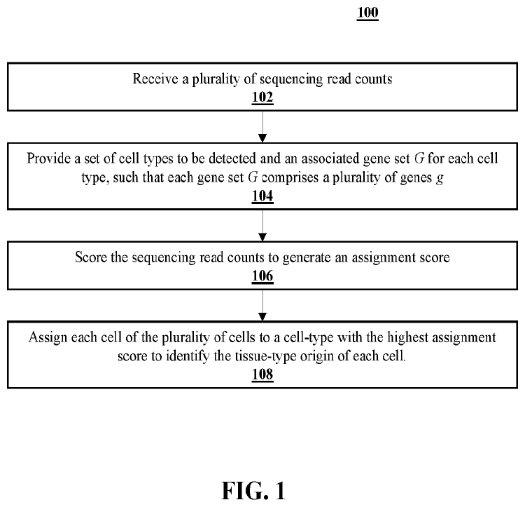

FIG. 1 is a flowchart describing sequential steps of the methods disclosed herein. In some embodiments, the method first includes receiving step 102 wherein a plurality of sequencing read counts are received by a computer-implemented system. In providing step 104, a set of cell types to be detected and an associated gene set G for each cell type is provided by the user, such that each gene set G comprises a plurality of genes g. In scoring step 106, the sequencing read counts are scored, as described in further detail below, to generate an assignment score. In step 108, each cell of the plurality of cells is assigned to a cell-type with the highest assignment score to identify the tissue-type origin of each cell.

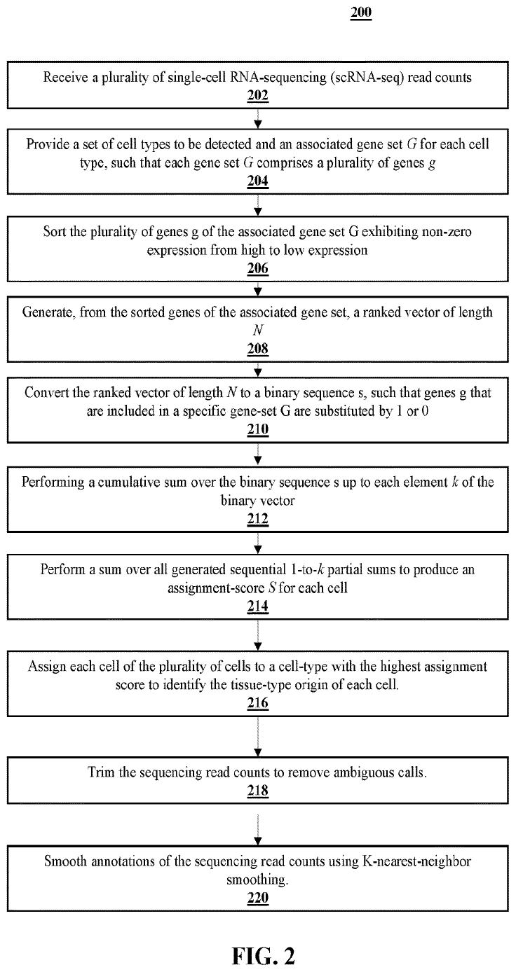

FIG. 2 is a flowchart describing sequential steps of the methods disclosed herein in further detail. In some embodiments, the method first includes receiving step 202 wherein a plurality of single-cell RNA-sequencing read counts are received by a computer-implemented system. In providing step 204, a set of cell types to be detected and an associated gene set G for each cell type is provided by the user, such that each gene set G comprises a plurality of genes g. In step 206, the plurality of genes g of the associate gene set G exhibiting non-zero expression are sorted from high to low expression. In step 208, a ranked vector of length N is generated from the sorted genes of the associated gene set. In step 210, the ranked vector of length Nis converted to a binary sequence s, such that genes g that are included in a specific gene-set G are substituted by either 1 or 0. In step 212, a cumulative sum is performed over the binary sequence s up to each element k of the binary vector. In step 214, a sum is performed over all generated sequential 1-to-k partial sums to produce an assignment-score S for each cell. In step 216, each cell of the plurality of cells is assigned to a cell-type with the highest assignment score to identify the tissue-type origin of each cell. In step 218, the sequencing read counts are optionally trimmed to remove ambiguous calls. In step 220, the annotations of the sequencing read counts are optionally smoothed using K-nearest-neighbor smoothing.

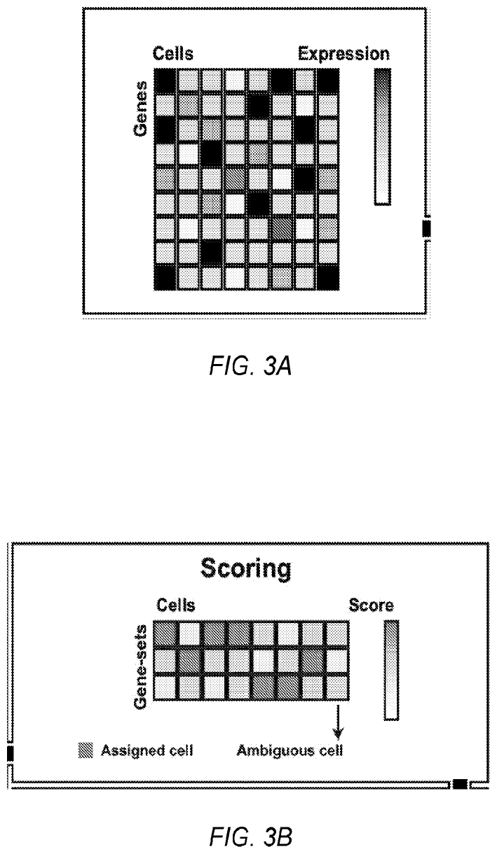

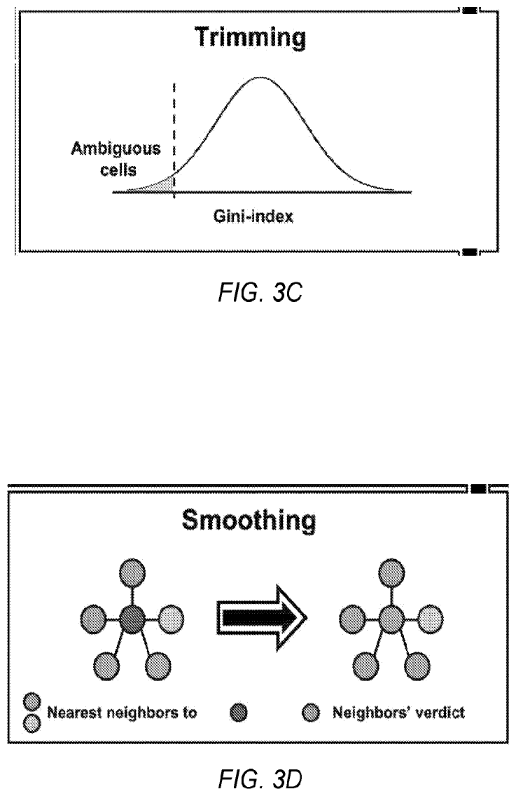

In some embodiments, the single-cell RNA-sequencing (scRNA-seq) data annotation pipeline disclosed herein is performed in three sequential steps: (1) scoring; (2) trimming; and (3) smoothing (FIGS. 3B-3D). Each step is described in further detail below. In some embodiments, the input to the methods disclosed herein consists of (1) a single cell transcriptomics dataset (scRNA-seq or snRNA-seq data), (2) a set of cell types to be detected, (3) and an associated gene set for each cell type. Methods relating to generating scRNA-seq data are reviewed in, e.g., Olsen T K and Baryawno N. Introduction to Single-Cell RNA Sequencing. Curr Protoc Mol Biol. 2018 Apr;122(1)., which is incorporated by reference herein in its entirety. As shown in FIG. 3A, expression of single genes (rows) are evaluated for each single cell (columns) in an experiment, based on scRNA-seq data. Methods for inferring gene expression levels from scRNA-seq data are reviewed in, e.g., Birnbaum K D. Power in Numbers: Single-Cell RNA-Seq Strategies to Dissect Complex Tissues. Annu Rev Genet. 2018 Nov. 23; 52:203-221, which is incorporated by reference herein in its entirety. The methods disclosed herein use a score-based procedure to infer the cell type of origin for each cell based on the provided gene sets. As shown in FIG. 3B, single cells (columns) are scored relative to provided gene sets (rows). In further detail, first, for a given cell, the method sorts non-zero expressed genes from high to low expression to generate a ranked vector. Then, this ranked vector (of length N) is converted to a binary sequence (s) so that genes (g) that are included in a specific gene-set (G) are substituted by 1, and 0 otherwise according to the following equation:

$$s = {\{ \begin{matrix}1 : {g}_{n} ∈ G \\ 0 : {g}_{n} ∉ G\end{matrix} \}}_{1 \leq n \leq N} .$$

Next, a partial cumulative sum is performed over the binary sequence up to each element “k” of the binary vector, followed by the sum over all generated sequential “1-to-k” partial sums, which results in assignment-score S for the given cell:

$$S = {\sum }_{k = 1}^{N} {\sum }_{n = 1}^{k} {s}_{n} .$$

This process is performed over all cells and gene sets (total M gene sets), transforming an input gene-by-cell expression matrix (number of genes×number of cells) into a gene-set-by-cell assignment-score matrix (number of gene sets×number of cells). Finally, each individual cell is assigned to the cell-type with the highest assignment-score:

$$max {\{ {S}_{m} \}}_{1 \leq m \leq M} .$$

### Trimming

In order to prevent misassignment when unknown cell types (unspecified in the marker gene-sets) are present, the method disclosed herein identifies cells that do not belong to any provided cell-type (FIG. 3C). In some embodiments, the methods disclosed herein utilize the Gini index as a measure of dispersion to identify ambiguous calls. According to the methods disclosed herein, first, for each cell the Gini index (see, e.g., Jiang L, et al. GiniClust: detecting rare cell types from single-cell gene expression data with Gini index. Genome Biol. 2016 Jul. 1; 17(1):144., which is incorporated by reference herein in its entirety) is calculated among its assignment scores, transforming the gene-set-by-cell assignment-score matrix to a distribution of Gini indexes (∈[0,1]). Then, cells with an index which is both outlier and less than 0.5 are designated as ambiguous. Cells with such ambiguous calls remain unclassified. In addition, if a cell does not express any of the specified markers or gains equal score across multiple cell-types, it will remain unclassified as well.

### Smoothing

Next k-nearest neighbor (kNN) smoothing (FIG. 3D) is performed. This step is designed to smoothen annotations by aggregating information from cells with similar genome-wide expression profiles (neighbors). Smoothing is performed by first identifying cells with a minimum of k nearest-neighbors. Then, if more than 50% of neighbors reach a consensus on their labels, the surrounded cell is relabeled according to the neighbors' consensus. Smoothing runs once over all cells.

### Additional Features

By default, the signature genes are expected to be highly expressed in one cell type compared to all other cell types. However, depending on the underlying data, these canonical markers may not be enough to segregate cell types with similar expression profiles. When this occurs, genes that are expected not to be detected in a specific cell type (e.g., CD8A in CD4 T cells) can be utilized to improve segregation. Therefore, genes that are known to be characteristically lowly expressed in one cell-type compared to the other cell types are introduced as the “negative markers”. In some embodiments, the methods disclosed herein incorporate negative markers by rewarding cells that do not express these markers and penalizing them otherwise. This series of steps can increase the dispersion among assignment scores, improving the accuracy of the method.

A sub-annotation strategy can be applied in the method based on a hierarchy of known cell types. In some embodiments, data undergo a first round of annotation at an intermediate hierarchy level (i.e., with broad cell type definitions). Subsequently, in some embodiments, each annotated group can be treated as a new separate dataset and annotated further with additional gene sets. In some embodiments, applying the methods disclosed herein as a successive hierarchical process improves the granularity and accuracy of the method, allowing for novel cell subtypes can be explored more efficiently.

Tissues and cells types for which the methods disclosed herein can be used to identify cell-types of single cells based on scRNA-seq data include, but are not limited to, peripheral blood mononuclear cells (PBMCs) including T cells and NK cells (TNK), monocytes and macrophages (MPh), B and plasma cells (BPC), neutrophils (Neut), erythrocytes (Eryth), megakaryocytes (Mega), and hematopoietic stem cells (HSC); macrophages and monocytes including classical (CD14+, CD16−), intermediate (CD14+, CD16+), and non-classical (CD14−, CD16+) monocytes; B cells including naïve B cells (B.Naive), memory B cells (MBCs), and plasma cells (PCs); heart tissue, kidney tissue, and lung tissue including cardiac fibroblasts (CFs), cardiac muscle cells (CMC), endothelial cells (Endo), hepatocytes (Hepa), macrophages (Mac), and smooth muscle cells (SMC); kidney tissue including B cells (B), endothelial cells (Endo), Epithelial cells (Epit), macrophages (Mac), and T and natural killer cell population (TNKs); and lung tissue including adventitial cells (AC), alveolar type 1 and type 2 cells (ATC), basal cells (Basal), basophils (Baso), B and Plasma cells (BPC), club cells (CC), endothelial (Endo), fibroblasts (Fibro), goblet, serous, and mucous cells (GSM), lung ciliated cells (LCC), mesothelial cells (MC), monocytes, dendritic cells, and macrophages (MPh), neutrophils (Neut), pericyte cells (PC), pulmonary ionocytes (PI), smooth muscle cells (SMC), T and natural killer cells (TNK).

In some embodiments, the tissue for which the methods disclosed herein can be used to identify cell-types of single cells based on scRNA-seq data include, but are not limited to prostate, lung, pancreas, cervical, renal, salivary gland uterine, gastric, thyroid, sinus, middle and inner ear, adrenal glands, appendix, hematopoietic system, bones and joints, spinal cord, breast, cerebellum, connective and soft tissue, corpus uteri, esophagus, eye, nose, eyeball, fallopian tube, extrahepatic bile ducts, mouth, intrahepatic bile ducts, kidney, appendix, larynx, lip, liver, lung and bronchus, lymph node, cerebral, spinal, nasal cartilage, retina, oropharynx, endocrine glands, female genital, ovary, penis and scrotum, pituitary gland, pleura, rectum, renal pelvis, ureter, peritoneum, salivary gland, skin, small intestine, testis, thymus, thyroid gland, tongue, unknown, urinary bladder, uterus, vagina, labia, or vulva tissue. In some embodiments, the sample comprises cells selected from the group consisting of adipose, adrenal cortex, adrenal gland, adrenal gland—medulla, appendix, bladder, blood, blood vessel, bone, bone cartilage, brain, breast, cartilage, cervix, colon, colon sigmoid, dendritic cells, skeletal muscle, endometrium, esophagus, fallopian tube, fibroblast, gallbladder, kidney, larynx, liver, lung, lymph node, melanocytes, mesothelial lining, myoepithelial cells, osteoblasts, ovary, pancreas, parotid, prostate, salivary gland, sinus tissue, skeletal muscle, skin, small intestine, smooth muscle, stomach, synovium, joint lining tissue, tendon, testis, thymus, thyroid, uterus, and uterus corpus. In some embodiments, the tissue is collected from a healthy subject. In some embodiments, the tissue is collected from a subject who is known, or is suspected to have, a cancer. In some embodiments, the tissue is collected from a solid or liquid tumor.

A list cell types and associated markers for designating gene sets can come from a variety of sources. In some embodiments, the methods disclosed herein include providing a list of user-designated gene-sets. In some embodiments, cell types and associated markers that are expected to be observed in a tissue under investigation are provided a priori. Sources for lists of tissue-specific markers include, for example, CellMarker (Zhang X, et al. CellMarker: a manually curated resource of cell markers in human and mouse. Nucleic Acids Res. 2019 Jan. 8; 47(D1):D721-D728. doi: 10.1093/nar/gky900. PMID: 30289549; PMCID: PMC6323899., which is incorporated by reference herein in its entirety) and PanglaoDB (Franzén O, et al. PanglaoDB: a web server for exploration of mouse and human single-cell RNA sequencing data. Database (Oxford). 2019 Jan. 1; 2019., which is incorporated by reference herein in its entirety). Expert annotated data are also available from databases including, for example, The Tabula Sapiens (Tabula Sapiens Consortium. The Tabula Sapiens: A multiple-organ, single-cell transcriptomic atlas of humans. Science. 2022 May 13;376(6594)., which is incorporated by reference herein in its entirety), which in some embodiments can be analyzed by the methods disclosed herein to define gene signatures. Signatures can be defined by a user's own experiments, including for example, CITE-seq (Stoeckius M, et al. Simultaneous epitope and transcriptome measurement in single cells. Nat Methods. 2017 Sep;14(9):865-868., which is incorporated by reference herein in its entirety), or cross-validation experiments.

The methods disclosed herein can be used as a stand-alone tool or as complementary to the other supervised methods. For example, the methods disclosed herein may be used in combination with scRNA-seq analysis workflows, for example, the Seurat package (Hao Y, et al. Integrated analysis of multimodal single-cell data. Cell. 2021 Jun. 24; 184(13):3573-3587., which is incorporated by reference herein in its entirety) to identify segregated cell populations and subsequently apply the methods disclosed herein to further refine the granularity of cell-type annotation.

### Computer Implementation of the Methods

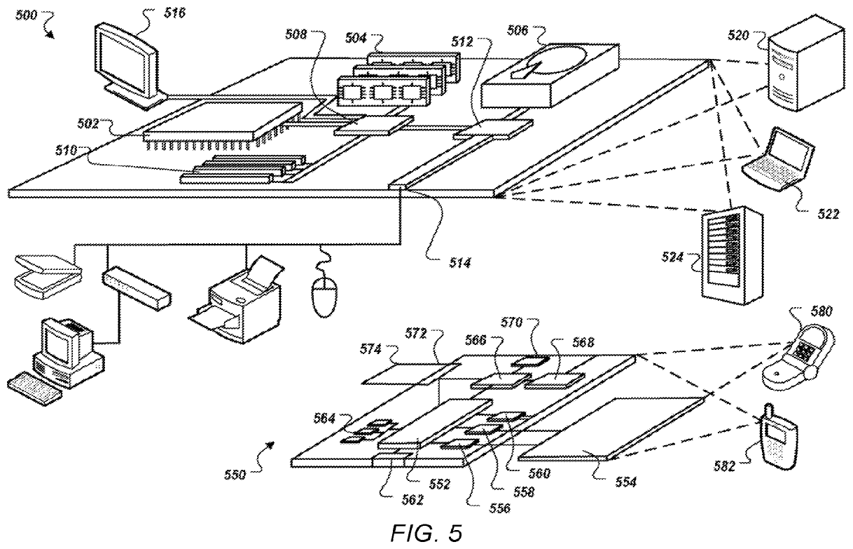

FIG. 5 is a diagram of computer system 500 components that can be used to implement methods for identifying a cell-type origin of each cell of a plurality of cells based on scRNA-seq data.

Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally, computing device 500 or 550 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be examples only, and are not meant to limit implementations of the methods and compositions described and/or claimed in this document.

Computing device 500 includes a processor 502, memory 504, a storage device 506, a high-speed interface 508 connecting to memory 504 and high-speed expansion ports 510, and a low speed interface 512 connecting to low speed bus 514 and storage device 506. Each of the components 502, 504, 506, 508, 510, and 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed interface 508. In other implementations, multiple processors and/or multiple buses can be used, as appropriate, along with multiple memories and types of memory. Also, multiple computing devices 500 can be connected, with each device providing portions of the necessary operations, e.g., as a server bank, a group of blade servers, or a multi-processor system.

The memory 504 stores information within the computing device 500. In one implementation, the memory 504 is a volatile memory unit or units. In another implementation, the memory 504 is a non-volatile memory unit or units. The memory 504 can also be another form of computer-readable medium, such as a magnetic or optical disk.

The storage device 506 is capable of providing mass storage for the computing device 500. In one implementation, the storage device 506 can be or contain a computer-readable medium, such as a floppy disk device, a hard disk device, an optical disk device, or a tape device, a flash memory or other similar solid-state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 504, the storage device 506, or memory on processor 502.

The high-speed controller 508 manages bandwidth-intensive operations for the computing device 500, while the low speed controller 512 manages lower bandwidth intensive operations. Such allocation of functions is only an example. In one implementation, the high-speed controller 508 is coupled to memory 504, display 516, e.g., through a graphics processor or accelerator, and to high-speed expansion ports 510, which can accept various expansion cards (not shown). In the implementation, low-speed controller 512 is coupled to storage device 506 and low-speed expansion port 514. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

Computing device 550 includes a processor 552, memory 564, and an input/output device such as a display 554, a communication interface 566, and a transceiver 568, among other components. The device 550 can also be provided with a storage device, such as a micro-drive or other device, to provide additional storage. Each of the components 550, 552, 564, 554, 566, and 568, are interconnected using various buses, and several of the components can be mounted on a common motherboard or in other manners as appropriate.

The processor 552 can execute instructions within the computing device 550, including instructions stored in the memory 564. The processor can be implemented as a chipset of chips that include separate and multiple analog and digital processors. Additionally, the processor can be implemented using any of a number of architectures. For example, the processor 510 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 550, such as control of user interfaces, applications run by device 550, and wireless communication by device 550.

Processor 552 can communicate with a user through control interface 558 and display interface 556 coupled to a display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can be provided in communication with processor 552, so as to enable near area communication of device 550 with other devices. External interface 562 can provide, for example, for wired communication in some implementations, or for wireless communication in other implementations, and multiple interfaces can also be used.

The memory 564 stores information within the computing device 550. The memory 564 can be implemented as one or more of a computer-readable medium or media, a volatile memory unit or units, or a non-volatile memory unit or units. Expansion memory 574 can also be provided and connected to device 550 through expansion interface 572, which can include, for example, a SIMM (Single In Line Memory Module) card interface. Such expansion memory 574 can provide extra storage space for device 550, or can also store applications or other information for device 550. Specifically, expansion memory 574 can include instructions to carry out or supplement the processes described above, and can also include secure information. Thus, for example, expansion memory 574 can be provided as a security module for device 550, and can be programmed with instructions that permit secure use of device 550. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that can be received, for example, over transceiver 568 or external interface 562.

Device 550 can communicate wirelessly through communication interface 566, which can include digital signal processing circuitry where necessary. Communication interface 566 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 568. In addition, short-range communication can occur, such as using a Bluetooth, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 can also communicate audibly using audio codec 560, which can receive spoken information from a user and convert it to usable digital information. Audio codec 560 can likewise generate audible sound for a user, such as through a speaker, e.g., in a handset of device 550. Such sound can include sound from voice telephone calls, can include recorded sound, e.g., voice messages, music files, etc. and can also include sound generated by applications operating on device 550.

The computing device 550 can be implemented in a number of different forms, as shown in the figure. For example, it can be implemented as a cellular telephone 580. It can also be implemented as part of a smartphone 582, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

Embodiments of the disclosure and all of the functional operations described in this specification can be implemented in digital electronic circuitry, or in computer software, firmware, or hardware, including the structures disclosed in this specification and their structural equivalents, or in combinations of one or more of them. Embodiments of the methods and compositions can be implemented as one or more computer program products, e.g., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the disclosure can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the methods, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

### Examples

The compositions and methods are further described in the following examples, which do not limit the scope of the compositions and methods described in the claims.

### Example 1: Cell-Type Identification Using Simulated Data

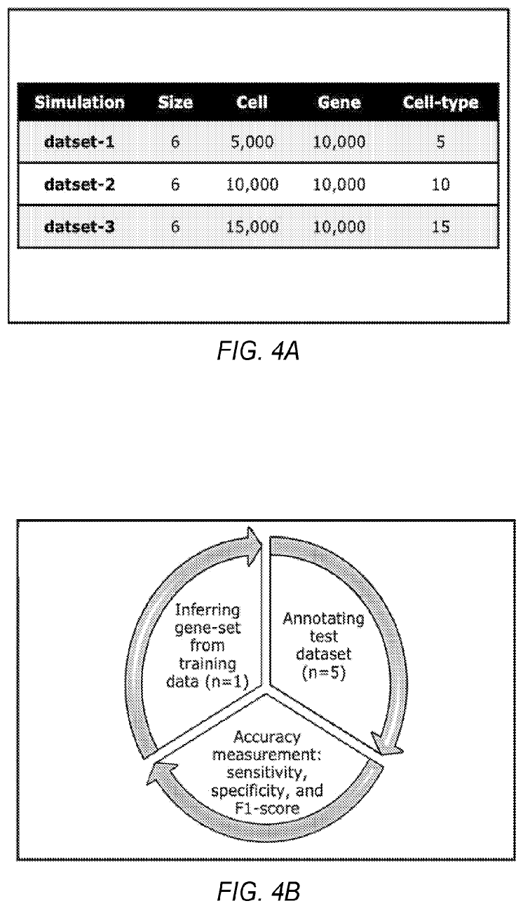

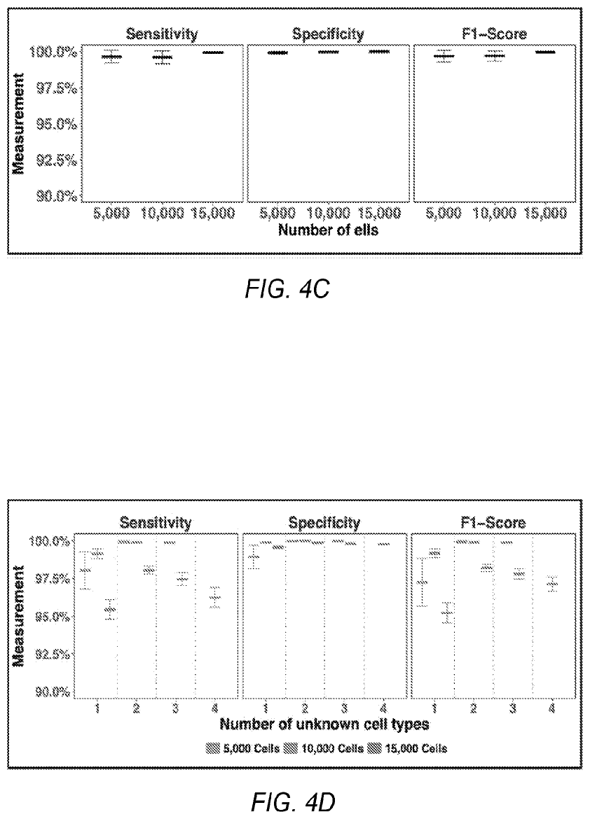

Performance of the cell-type identification method was benchmarked using simulated data, where cell-types were known apriori. The R-package Splatter (Zappia L, et al. Splatter: simulation of single-cell RNA sequencing data. Genome Biol. 2017 Sep. 12; 18(1):174.) was used with default parameters to create three sets of simulated scRNA-seq data of increasing size and complexity: (1) six datasets with 5,000 cells divided into five cell-types; (2) six datasets with 10,000 cells divided into ten cell-types; (3) six datasets with 15,000 cells divided into 15 cell-types. The proportion of cells in each cell-type group were randomly sampled from a uniform distribution. Each simulated dataset included 10,000 genes as shown in FIG. 4A.

For benchmarking using simulated data, the cell-type identification method used cell type-specific markers as the input. In order to infer cell type-specific markers from simulated data, a systematic approach was designed. First, one simulated dataset was selected as a training dataset (1 training+5 testing). Marker gene-sets were inferred from the training data by performing differential expression analysis using Wilcoxon rank sum test, using the FindAllMarkers function from Seurat R-package (Hao Y, et al. Integrated analysis of multimodal single-cell data. Cell, 2021 Jun 24;184(13):3573-3587.e29.) with a minimum fraction of 0.1 cells expressing a given gene and at least 0.5-fold difference (log-scale) between the two groups of cells. The 100 top-ranked marker genes for each cell-type were used as the input for annotation of remaining five testing datasets. This approach provided a benchmark of 30 trials for each set of simulated data, as shown in FIG. 4B.

As shown in FIG. 4C, the performance of the cell-type identification method using simulated data was quantified using the measures of sensitivity (SEN), specificity (SPC), and F1-score by the fractions TP/(TP+FN), TN/(TN+FP), and 2×TP/(2×TP+FP+FN), respectively, where:
1. True positive (TP) was defined by the number of cell type-related pairs that were correctly identified,
2. False positive (FP) was defined by the number of unrelated cell pairs that were incorrectly identified as cell type-related,
3. True negative (TN) was defined by the number of unrelated cell pairs that were correctly identified as unrelated, and
4. False negative (FN) was defined by the number of cell type-related pairs that were incorrectly identified as unrelated.

As shown by the plots of FIG. 4C, the cell-type identification method inferred the simulated cell type assignments with average sensitivity, specificity, and F1-score of above 99% across all trials.

The performance of the cell-type identification method was evaluated with respect to discovering novel cell types (i.e., unclassified cell types). This task was performed by omitting a subset of the marker gene sets and applying the cell-type identification method to retrieve the missing cell types. One training dataset was chosen at random, and between 1-4 cell types from the training data at random: 1-2 cell types from data with 5 cell types; 1-3 cell types from data with 10 cell types; and 1-4 cell types from data with 15 cell types. Next, marker gene-sets were inferred from the training data by performing differential expression analysis as discussed above. Last, the cell-type identification method was benchmarked using remaining simulated data. This procedure was repeated 100 times for each cell type removal. Cell types were removed before marker gene selection to ensure that marker genes were being selected with no knowledge of unknown cell types. As shown in FIG. 4D, the cell-type identification method inferred the unknown cell types with average sensitivity, specificity, and F1-score values of above 95% across all trials.

The cell-type identification method is not sensitive to the inclusion of marker gene sets for cell type not occurring in the dataset. Because the cell-type identification method is a single cell-based algorithm, the inclusion of a marker set for which no cells are found does not affect the score of the other marker gene sets, and therefore it does not impact the annotation quality of cell types present in the dataset.

### Example 2: Cell-Type Identification Using Experimental Data

The performance of the cell-type identification method disclosed herein was evaluated by annotating experimental scRNA-seq data from multiple human tissues from the published Tabula Sapiens Consortium (Tabula Sapiens Consortium. The Tabula Sapiens: A multiple-organ, single-cell transcriptomic atlas of humans. Science. 2022 May 13;376(6594):eab14896.). 50,115 peripheral blood mononuclear cells (PBMCs) were extracted from the Tabula data and classified using a hierarchy of known immune cell-types. First, a marker gene sets was generated, specifying cell types at a moderate immunophenotype granularity, namely: T cells and NK cells (TNK), monocytes and macrophages (MPh), B and plasma cells (BPC), neutrophils (Neut), erythrocytes (Eryth), megakaryocytes (Mega), and hematopoietic stem cells (HSC). Marker genes for T cells and NK cells included CD4, CD3D, CD3E, CD3G, IL7R, CD8A, CD8B, FOXP3, TIGIT, CD27, NCAM1, KLRF1, GNLY, NKG7, and TNFRSF18. Marker genes for monocytes and macrophages included CD4, MS4A7, VCAN, CD14, FCN1, LYZ, FCGR3A, ITGAM, MARCO, CD1C, and ITGAX Marker genes for B and plasma cells included CD79A, CD79B, MS4A1, CD19, CD24, CD37, CD72, TNFRSF17, MZB1, CR2, BCL11A, FCRL1, FCRL2, FCRL5, SP1B, FCRLA, and JCHAIN. Marker genes for neutrophils included S100A8, S100A9, IFITM2, FCGR3B, MS4A2, CPA3, TPSAB1, FCER1A, and MPO. Marker genes for erythrocytes included HBB, HBA1, and HBA2. Marker genes for megakaryocytes included PPBP, PF4, and ITGA2B. Marker genes for hematopoietic stem cells included CD34, PROM1, SPINK2, HOPX, CLEC9A, SOX4.

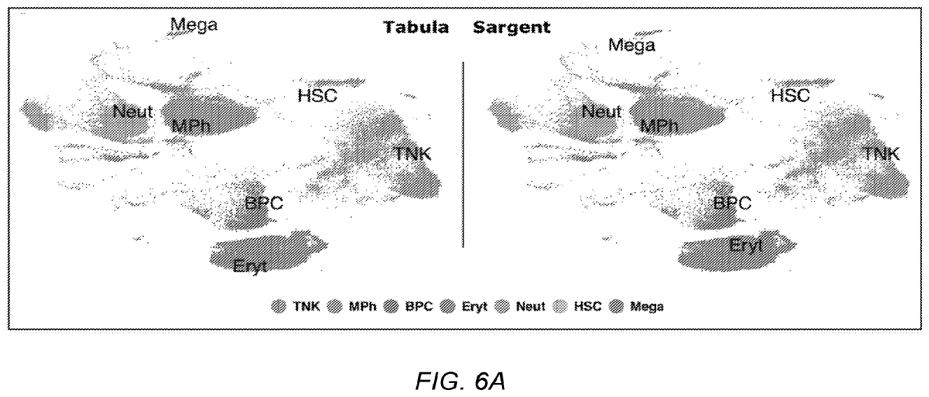

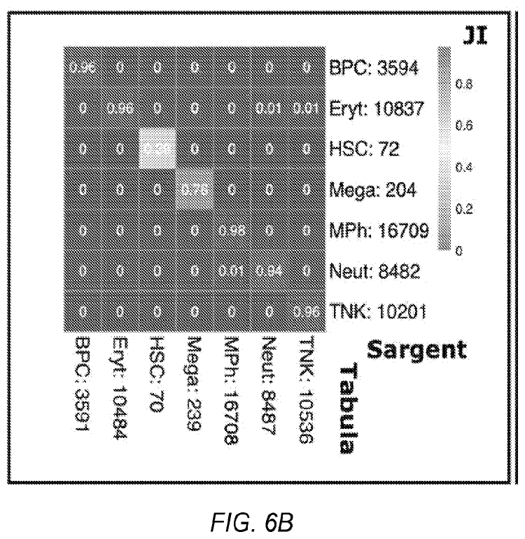

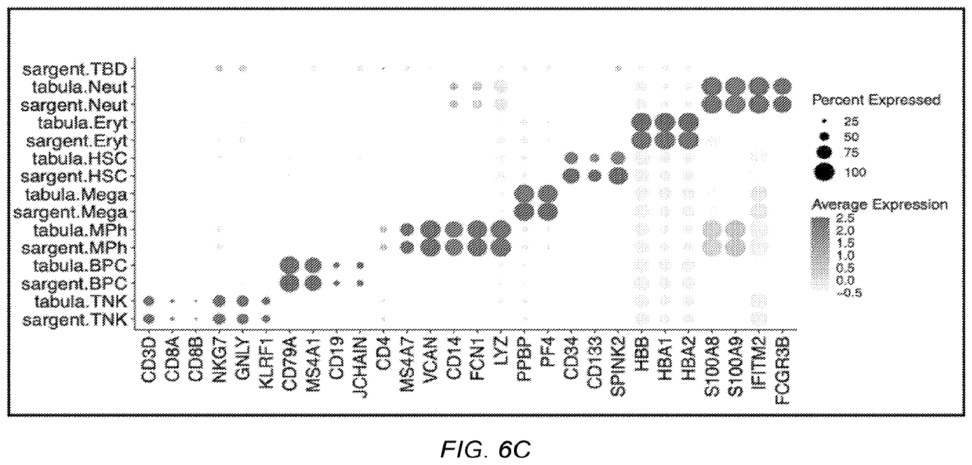

As shown in FIG. 6A, the cell-type identification method assigned cells to the correct type (the expert annotated type), with 98% accuracy across all cell types. As shown in FIG. 6B, high assignment accuracy was also demonstrated by the Jaccard Index (JI): 98% among MPhs; 96% among TNKs, BPCs, and Eryts; 94% among Neuts; and 76% among Megas. A relatively lower JI was achieved among HSCs: 39%. As shown in FIG. 6C, the expression level of the HSCs' canonical markers, CD34, CD133, and SPINK2, indicated that cells annotated by the cell-type identification method disclosed herein are more likely to be HSCs than the manually annotated cells. Only 16 out of more than 50,000 cells were not assigned to a cell type by the cell-type identification method as they did not exhibit any expression of all the immunophenotypes' canonical markers.

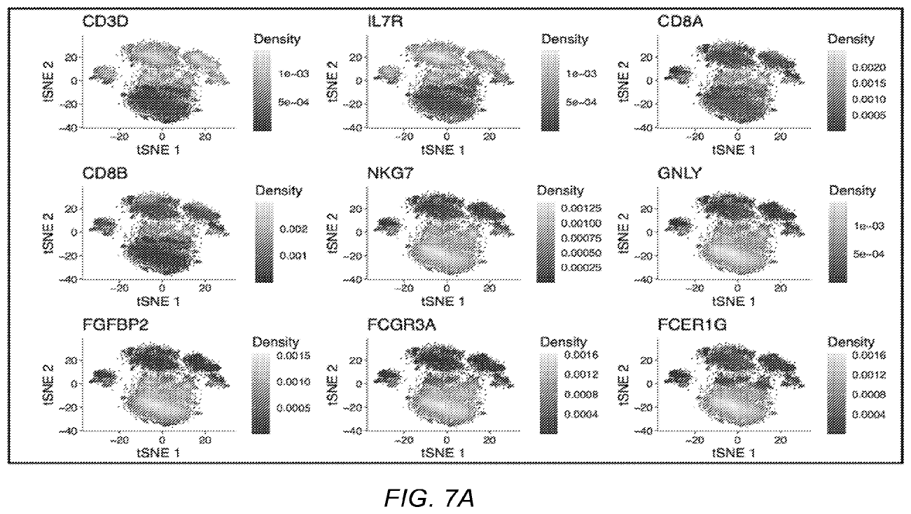

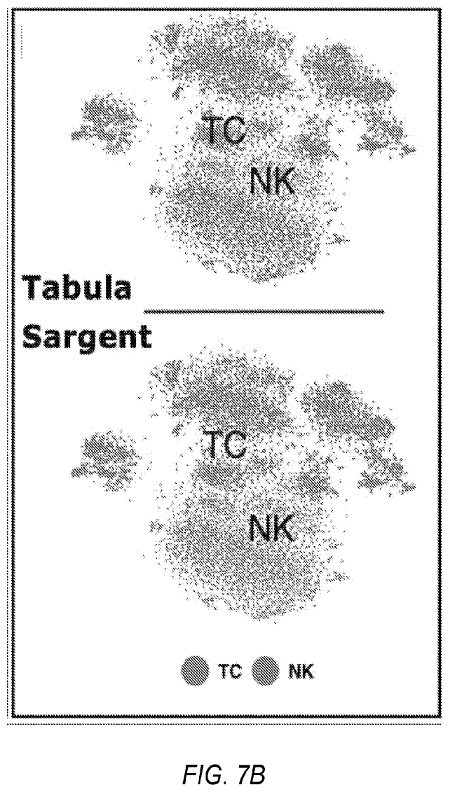

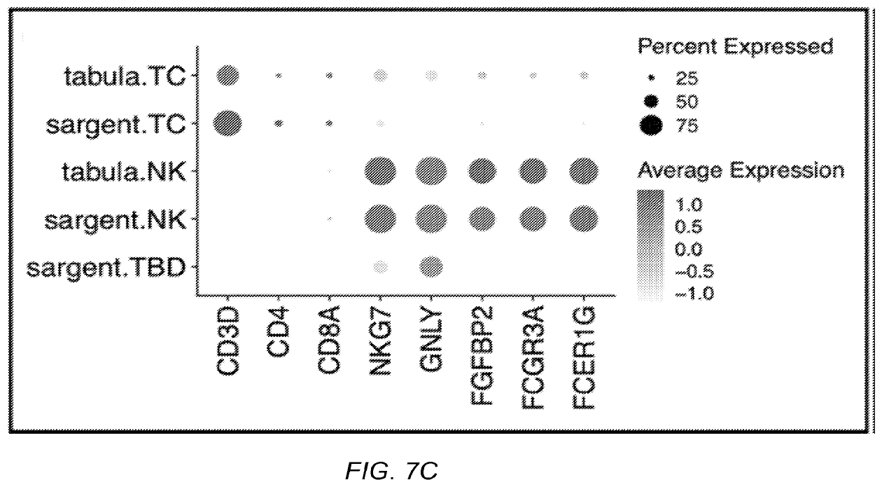

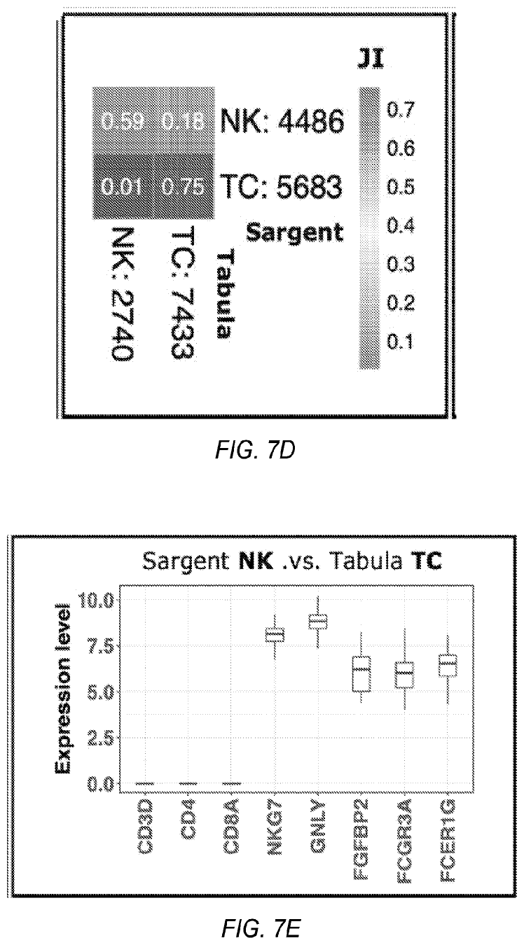

Performance of the cell-type identification method was evaluated by increasing the granularity of annotated cell types within the PBMC cell population. 10,173 cells that were jointly annotated as T cells or natural killer cells (TNK) by the cell-type identification method disclosed herein and the Tabula study were retrieved from the Tabula consortium data. Next, a list of T and NK (natural killer) cells canonical markers was curated, with the canonical markers shown in FIG. 7A. Marker genes for T cells included CD4, CD3D, CD3E, CD3G, IL7R, CD8A, CD8B, FOXP3, TIGIT, CD27, NKG7, and GNLY. Marker genes for natural killer cells included KLRF1, KLRC1, KLRC2, NCAM1, FGFBP2, FCGR3A, CX3CR1, NCR1, FCER1G, KDELC1, NKG7, GNLY, KLRD1, and CD7.

As shown in FIG. 7B, the cell-type identification method annotated 5,683 T cells and 4,486 NK cells with a clear separation in t-SNE space, with only 4 cells remaining unlabeled (FIG. 7C). As shown in FIG. 7D, the Jaccard Index similarity between the cell-type identification method and the Tabula study was 75% among T cells and 59% among NK cells. A mixed population (18% JI) of 1.795 cells (17% of TNKs) was identified by the cell-type identification method disclosed herein as NK cells and by the Tabula study as T cells (FIG. 7D). This mixed-annotation population was explored to determine which of the two annotations was more plausible. The expression level of T-cell canonical markers was examined including CD3D, CD4, and CD8A. As shown in FIG. 7E, this mixed population did not express the T-cell canonical markers. In contrast, they highly expressed cytotoxic markers like NKG7, GNLY, FGFBP2, FCGR3A, and FCER1G. Therefore, the NK cell annotation as predicted by the cell-type identification method disclosed herein was more plausible than the T cells annotation of the Tabula study.

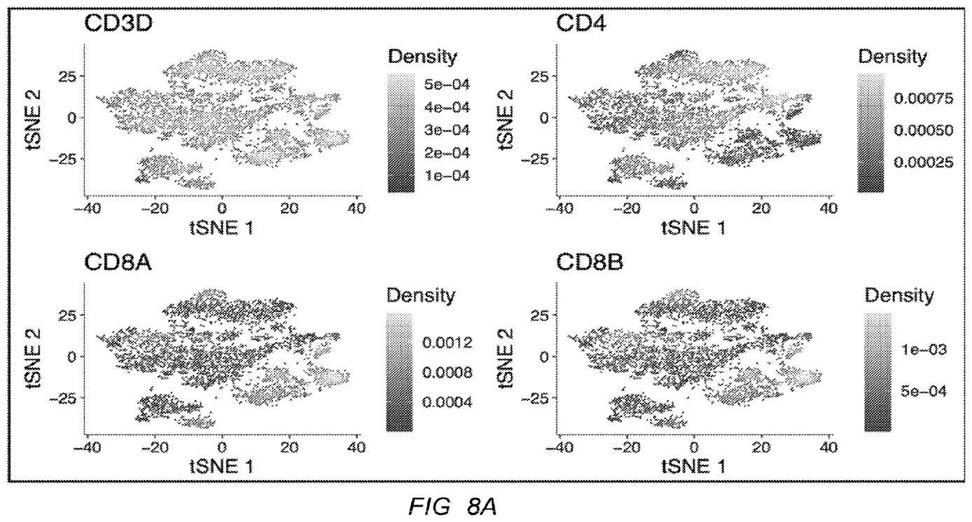

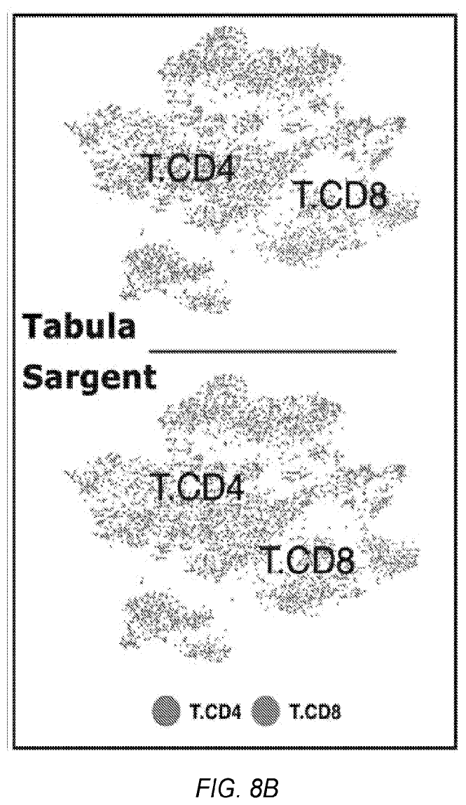

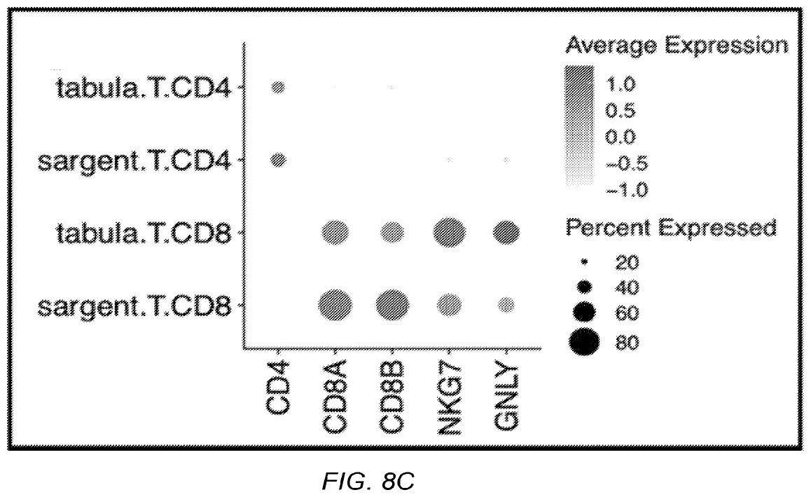

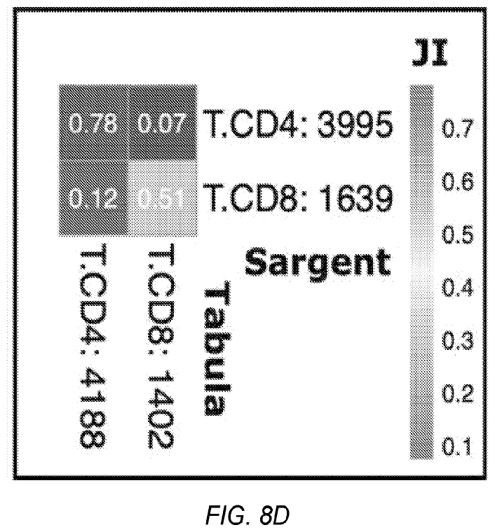

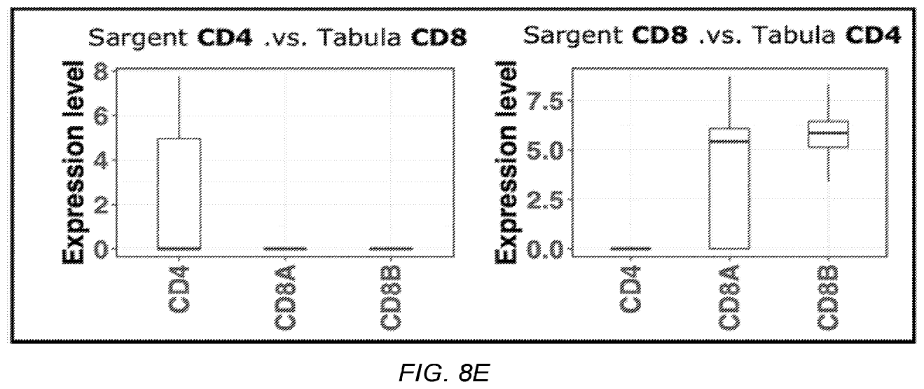

Next, resolution of the cell-type identification method was increased by focusing only on T cells. 5,634 cells that were jointly annotated as T cells by the cell-type identification method disclosed herein and the Tabula study were retrieved. A list of CD4+ and CD8+ T-cell canonical markers was curated (FIG. 8A) Marker genes for CD4+ T cells included CD3D, IL7R, and CD4, and negative marker genes for CD4+ T cells included CD8A and CD8B. Marker genes for CD8+ T cells included CD3D, IL7R, CD8A, and CD8B, and negative marker genes for CD8+ T cells included CD4.

As shown in FIGS. 8B and 8C, the cell-type identification method annotated 3,995 CD4+ T and 1,639 CD8+ T cells. As shown in FIG. 8D, the Jaccard Index similarity between the cell-type identification method and the Tabula study was 78% among CD4+ T and 51% among CD8+ T cells. In addition, a mixed population (17% of TCs) was observed among cells annotated by the cell-type identification method and the Tabula study, comprising 376 cells (7% JI) annotated as CD4+ T by the cell-type identification method but CD8+ T cells by the Tabula study, and 605 cells (12% JI) vice versa (FIG. 8D). These two mixed populations were explored to determine the more plausible annotation. The expression level of T-cell canonical markers, CD4, CD8A, and CD8B was evaluated. As shown in FIG. 8E, gene expression of these markers indicated that cells annotated as CD4+ T cells by the cell-type identification method expressed CD4 marker, and not CD8A and CD8B (left-panel). In contrast, cells annotated as CD8+ T cells by the Tabula study did not express CD8A and CD8B markers. Furthermore, as shown in FIG. 8E, cells annotated as CD8+ T cells by the cell-type identification method expressed CD8A and CD8B markers, and not CD4 (right-panel), while cells annotated as CD4+ T cells by the Tabula study expressed a relatively high level of CD8A and CD8B markers, and not CD4 marker. These results indicate that annotation by the cell-type identification method disclosed herein was more plausible than the Tabula study for the CD4+ and CD8+subpopulations.

Next, annotation of macrophages and monocytes by the cell-type identification method disclosed herein was evaluated. Performance of the cell-type identification method was benchmarked by annotating macrophages and the three major monocyte populations: classical (CD14+, CD16−), intermediate (CD14+, CD16+), and non-classical (CD14−, CD16+) monocytes. 16,504 cells that were jointly annotated as MPh by both the cell-type identification method and the Tabula study were retrieved. A list of macrophages and monocytes canonical markers was compiled. Marker genes for macrophages included ITGAM, ITGAX, CD68, MRC1, FCGR1A, and FCGR2A, and negative marker genes for macrophages included CD14, FCGR3A, CDKN1C, RHOC, LYZ, andMS4A7. Marker genes for classical (CD14+, CD16−) monocytes included CD14 and LYZ, and negative marker genes for classical monocytes included FCGR3A, MS4A7, CD68, CDKN1C, and RHOC. Marker genes for intermediate (CD14+, CD16+) monocytes included CD14 and FCGR3A, and negative marker genes for intermediate monocytes included CD68. Marker genes for non-classical (CD14−, CD16⁺) monocytes included FCGR3A and MS4A7, and negative marker genes for non-classical monocytes included CD14, LYZ, and CD68.

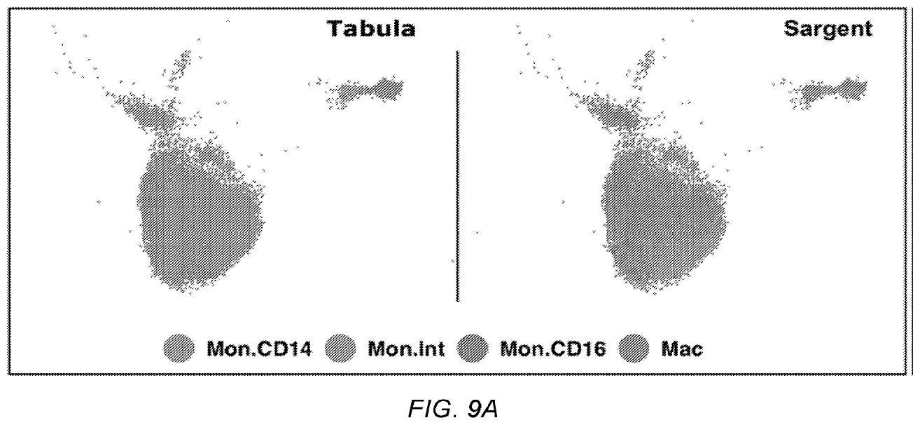

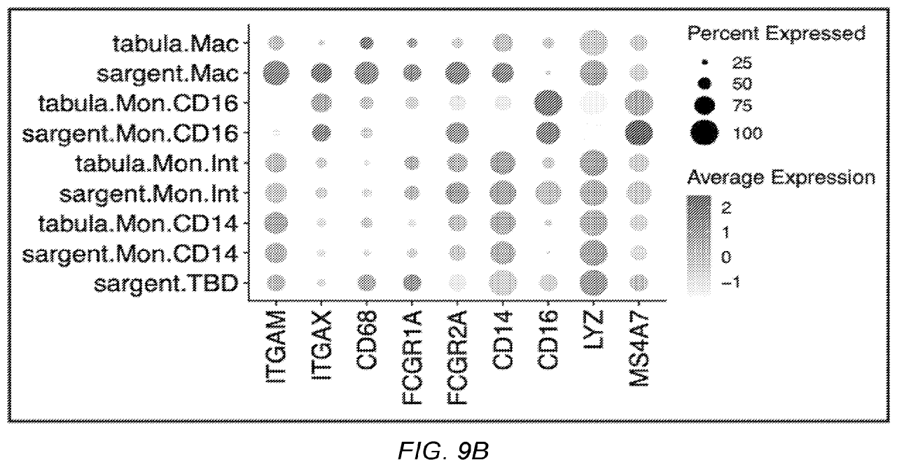

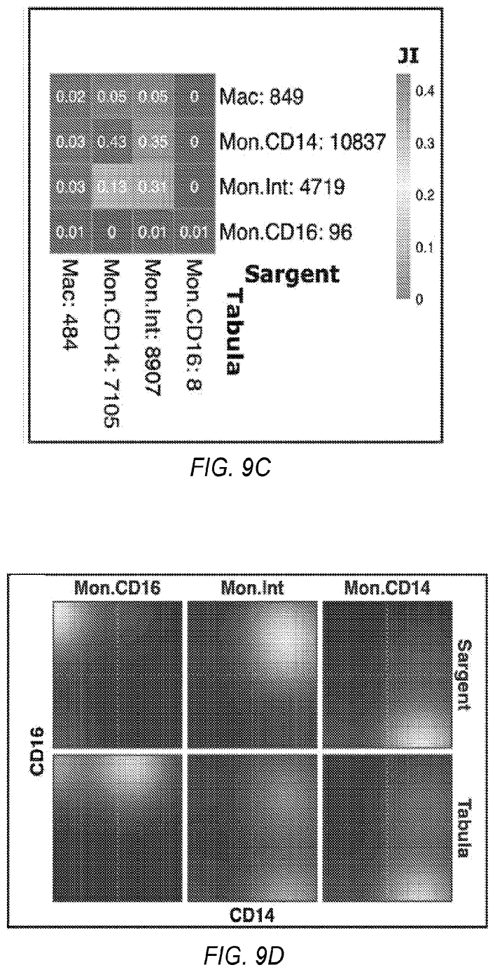

As shown in FIGS. 9A and 9B, the cell-type identification method annotated 894 macrophages, 10,837 classical monocytes, 4,719 intermediate monocytes, 96 non-classical monocytes, and 3 unlabeled. As shown in FIG. 9C, the similarity between the cell-type identification method disclosed herein and the Tabula study annotation measured by Jaccard Index indicated a poor agreement and, as for T and NK cells above, accuracy in each annotation was evaluated. First, the expression level of macrophages canonical markers including ITGAM, ITGAX, CD68, FCGR1A, and FCGR2A was evaluated. This evaluation indicated that macrophages annotated by the cell-type identification method disclosed herein expressed a higher level of the canonical markers than the Tabula study annotated macrophages (FIG. 9B). As shown in FIG. 9D, further inspection in the monocyte populations showed that the cell-type identification method disclosed herein achieved a clear segregation among classical, intermediate, and non-classical monocytes (top panels). In contrast, cells annotated by the Tabula study showed a mixed population indicating failure in annotation (bottom panels). Therefore, these data indicate that cells annotated by the cell-type identification method disclosed herein were more likely to be macrophages or monocytes than manually annotated cells were.

Next, performance of the cell-type identification method disclosed herein was benchmarked on the B cell population comprising naïve B cells (B.Naive), memory B cells (MBCs), and plasma cells (PCs). 3,525 cells that were jointly annotated as BPC by the cell-type identification method and Tabula were retrieved. A list of canonical markers highlighting memory, naïve, and plasma states was curated. Marker genes for naïve B cells included CD72, CD69, CD24, IGHM, and IGHD, and negative marker genes for naïve B cells included MZB1, PRDM1, XBP1, IGHG1, IGHG2, IGHG3, IGHA1, CD27, MME. Marker genes for memory B cells included CD72, CD69, CD24, IGHG1, IGHG2, IGHG3, IGHA1, IGHA2, IGHE, MME, and CD27, and negative marker genes for memory B cells included MZB1, PRDM1, XBP1, IGHM, and IGHD. Marker genes for plasma cells included PRDM1, XBP1, JCHAIN, MZB1, and negative marker genes for plasma cells included CD72, CD69, and CD24.

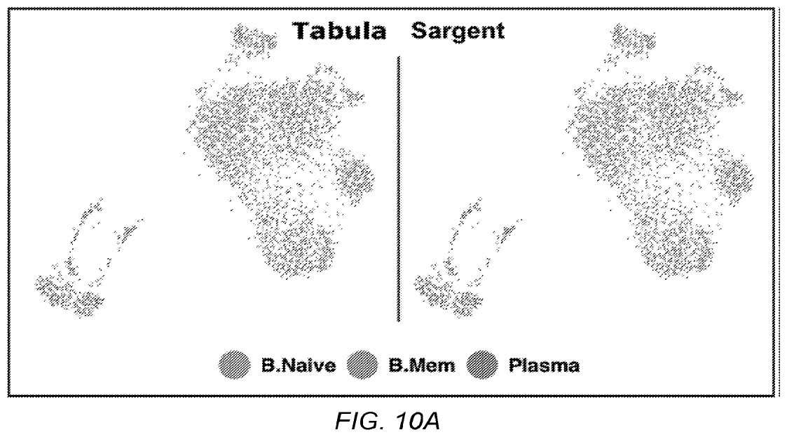

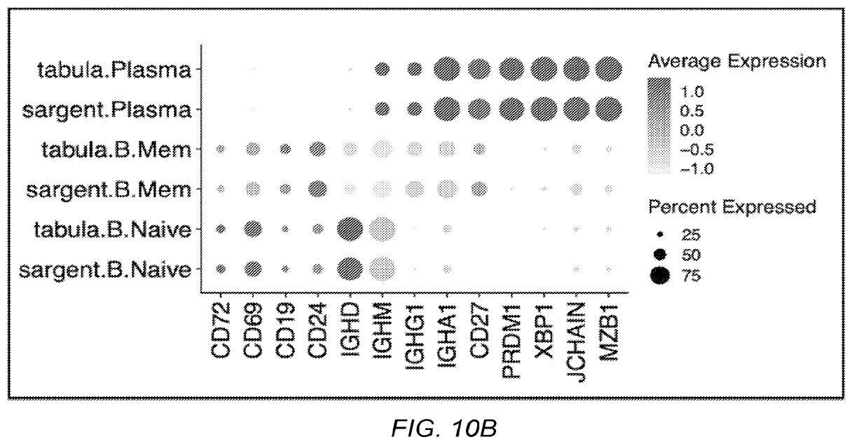

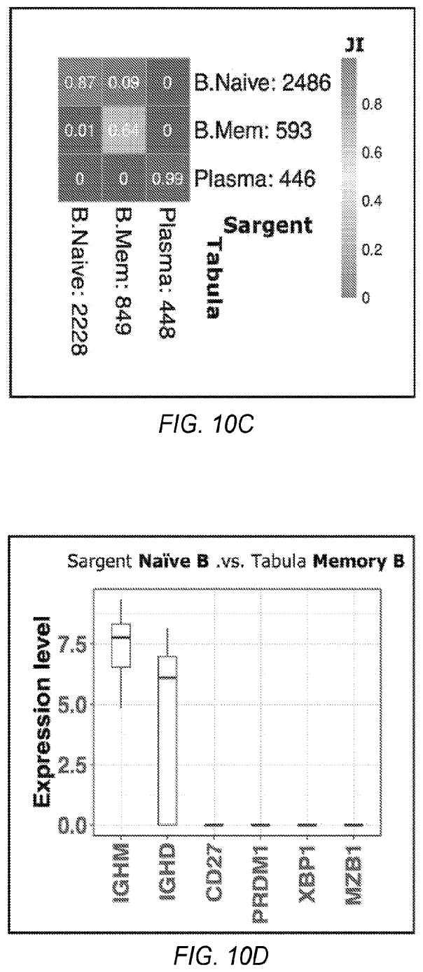

As shown in FIGS. 10A and 10B, the cell-type identification method annotated 2,486 NBCs, 593 MBCs, and 446 PCs. As shown in FIG. 10C, the Jaccard Index similarity between the cell-type identification method and the Tabula study annotation was 87% similarity among NBCs, 64% similarity among MBCs, and 99% for PCs. A mixed population (9% JI) of 284 cells (8% of BPCs) was identified to comprise of cells annotated as NBCs by the cell-type identification method and as MBCs by the Tabula study (FIG. 10C). When the expression level of BCs canonical markers including naïve markers IGHM and IGHD was examined, and activation marker CD27, this population highly expressed IGHM and IGHD and lacked the expression of CD27, as shown in FIG. 10D. Therefore, these data indicate that annotation of this population as NBC's by the cell-type identification method is more likely to be correct than the Tabula annotation.

Next, performance of the cell-type identification method in assigning cell types of additional three human tissues (heart, kidney, and lung) was evaluated relative to the manual cell type annotations from the Tabula study. The manual annotation of the human heart tissue comprised 6 cell types including: cardiac fibroblasts (CFs), cardiac muscle cells (CMC), endothelial cells (Endo), hepatocytes (Hepa), macrophages (Mac), and smooth muscle cells (SMC). A set of marker genes from the literature was compiled to recognize these cell types in the dataset. Marker genes for cardiac fibroblasts included DCN, GSN, PDGFRA, COL1A1, C7, LUM, and TCF21. Marker genes for cardiac muscle cells included NPPA, MYL7, MYL4, TNNT2, and MYH6. Marker genes for endothelial cells included VWF, PECAM1, CDH5, FABP4, AQP7, NPR3, PLVAP, and KDR. Marker genes for hepatocytes included TAGLN, AFP, KRT8, KRT18, HNF4A, LRP5, FGFR4, ASGR1, UCP2, HHEX, FOXA1, FOXA2, FOXA3, CDH1, FGA, FGB, FGG, APOA2, APOC3, and AMBP. Marker genes for macrophages included CD163, C1QA, TAGLN, MARCO, MRC1, ITGAM, MS4A7, CD14, FCGR3A, CD4, S100A8, S100A9, C1QC, GPR183, LYZ, ITGAX, VSIG4, FCER1G, TLR2, CD68, PTPRC, CSF1R, ITGAX, VSIG4, CD86, DUSP1, and DUSP2. Marker genes for smooth muscle cells included MYH11, TAGLN, ACTA2, CALD1, and NOTCH3.

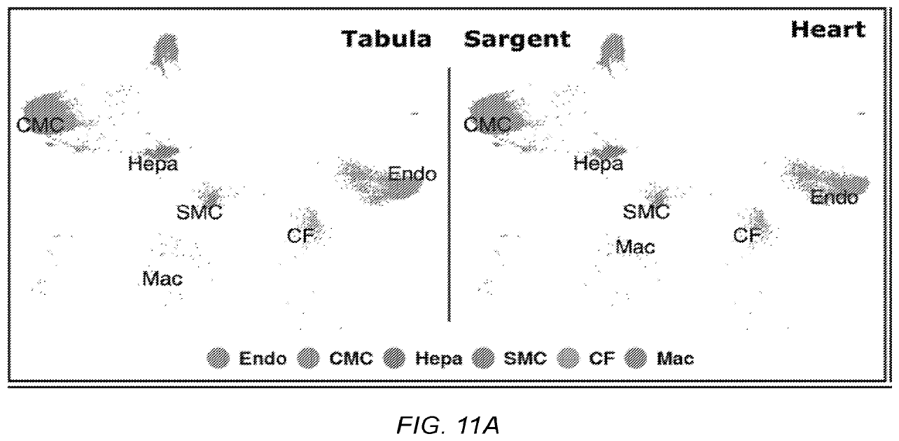

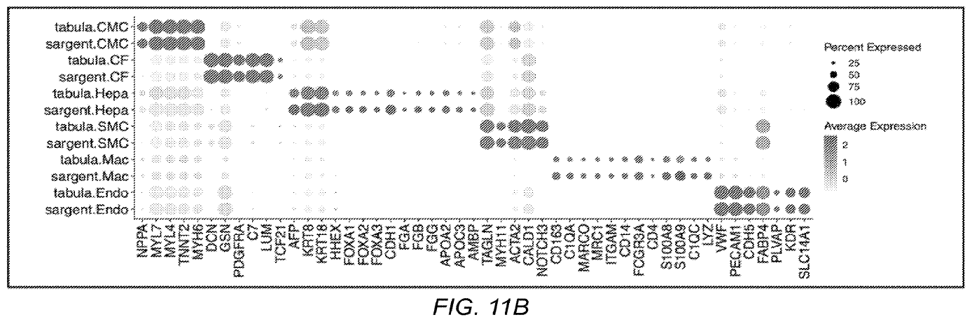

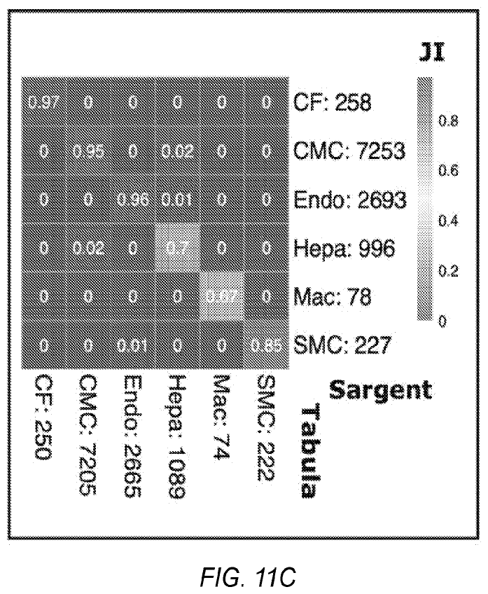

As shown in FIGS. 11A and 11B, the cell-type identification method disclosed herein assigned cells with 96% similarity with the manually annotated cells over all six heart tissue cell types. As shown in FIG. 11C, High assignment similarity between the cell-type identification method and the manual annotation of the Tabula study was shown at individual cell type level as measured by Jaccard Index: 97% for CFs; 96% for Endo cells; 95% for CMCs; 85% for SMCs; 70% similarity Hepa cells; and 67% for Macs.

Next, performance of the cell-type identification method disclosed herein was evaluated for human kidney tissue, which included 9,461 cells. The original annotation comprised of 5 cell types: B cells (B), endothelial cells (Endo), Epithelial cells (Epit), macrophages (Mac), and T and natural killer cell population (TNKs), for each of which a set of marker genes was compiled. Marker genes for kidney B cells included CD79A, CD79B, MS4A1, CD19, and JCHAIN. Marker cells for kidney endothelial cells included TGFBR2, NOTCH4, ADGRL4, EMCN, ENG, PECAM1, PLVAP, TEK, KDR, EPCAM, GJA5, CDH5, SEMA3G, PTPRB, and SLC14A1. Marker genes for kidney epithelial cells included ANPEP, AQP1, SLC13A3, SLC16A9, SLCI7A3, SLC22A7, and SLC22A8. Marker genes for kidney macrophages included CD4, MS4A7, VCAN, CD14, FCN1, LYZ, FCGR3A, ITGAM, MARCO, CD1C, and ITGAX. Marker genes for kidney T and natural killer cells included CD4, CD3D, CD3E, CD3G, IL7R, CD8A, CD8B, FOXP3, TIGIT, CD27, NCAM1, KLRF1, GNLY, NKG7, and TNFRSF18.

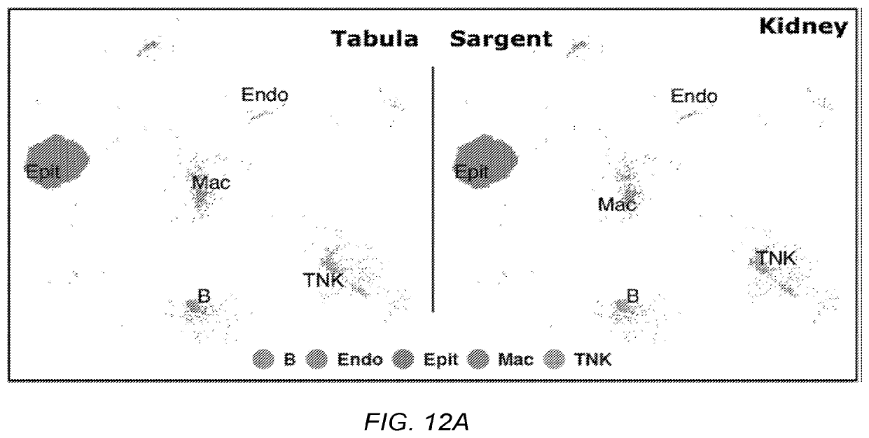

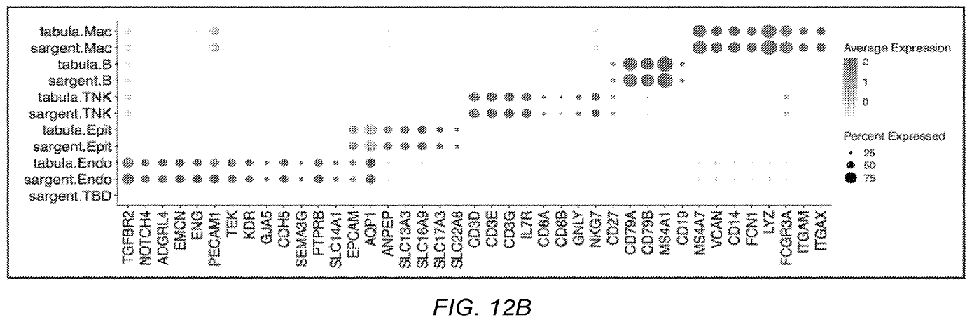

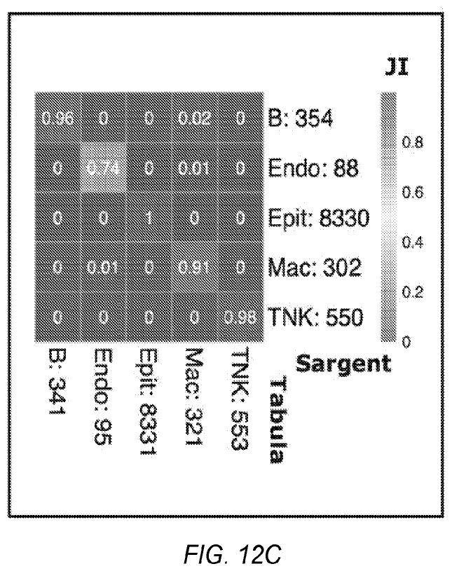

As shown in FIGS. 12A and 12B, the cell-type identification method was able to achieve a high similarity, 99%, with the manual annotations of the Tabula study over all cell types, with only 17 cells remained unlabeled (FIG. 12B). As shown in FIG. 12C, high assignment similarity between the cell-type identification method and the manual annotation of the Tabula study was demonstrated by the high Jaccard Index: 100% for Epit cells; 98% for TNKs; 96% for B cells; 91% for Macs; and 74% for Endo cells.

Finally, performance of the cell-type identification method disclosed herein was evaluated for complex human lung tissue (35,682 cells from Tabula Sapiens study). The original annotation included 17 cell types: adventitial cells (AC), alveolar type 1 and type 2 cells (ATC), basal cells (Basal), basophils (Baso), B and Plasma cells (BPC), club cells (CC), endothelial (Endo), fibroblasts (Fibro), goblet, serous, and mucous cells (GSM), lung ciliated cells (LCC), mesothelial cells (MC), monocytes, dendritic cells, and macrophages (MPh), neutrophils (Neut), pericyte cells (PC), pulmonary ionocytes (PI), smooth muscle cells (SMC), T and natural killer cells (TNK). A set of marker genes was compiled from the literature to detect and annotate these cell types in the dataset. Marker genes for adventitial cells included CD34, ANPEP, UAP1, VIT, MFAP5, PCOLCE2, GFPT2, DPT, PDGFRA, LUM, and SCARA5. Marker genes for alveolar type 1 and type 2 cells included AGER, CAV1, CLDN18, CAV2, ALOX15B, LRRK2, ROS1, SFTPA1, and CSF3R. Marker genes for basal cells included AQP3, KRT5, KRT14, KRT15, TP63, DAPL1, MIR205HG, EYA2, CYP24A1, and KRT17. Marker genes for basophils included CD63, CD203C, CD123, CLC, MS4A3, TCN1, CPA3, HDC, GATA2, MS4A2, IL4, GCSAML, and TPSAB1. Marker genes for B and plasma cells included CD79A, CD79B, MS4AJ, JCHAIN, TNFRSF17, DERL3, FCRL5, MZB1, VPREB3, IGHA1, IGHM, IGHG1, CD27, SLAMF7, EAF2, and CD38. Marker genes for club cells included SCGB3A2, CYP2B7P, MGP, SFTPC, and SFTPD. Marker genes for lung endothelial cells included CD34, EGFL7, EMCN, FLT1, KDR, TEK, VWF, ACKR1, and CA4. Marker genes for lung fibroblasts included COL3A1, COL5A2, FN1, LUM, LRP1, PDGFRA, TCF21, and SCARA5. Marker genes for goblet, serous, and mucous cells included MUC5B, MUC5AC. SPDEF, SCGB1A1, SCGB3A1, BP1FB1, LTF, ANPEP, EPCAM, IL10, and LTF. Marker genes for lung ciliated cells included FOXJ1, TUBB1, TP73, CCDC78, and CAPS. Marker genes for lung mesothelial cells included MSLN, UPK3B, WT1, CALB2, VCAM1, MEDAG, GAS1, HAS1, C1S, C2, and CFB. Marker genes for lung monocytes, dendritic cells, and macrophages included CD4, MS4A7, VCAN, FCN1, FCGR3A, ITGAM, CD1C, ITGAX, LILRB2, and CD14. Marker genes for lung neutrophils included S100A8, S00A12, FCGR3B, LILRA5, and GOS2. Marker genes for lung pericyte cells included KCNK3, CDH6, COX4I2, NDUFA4L2, PDGFRB, CSPG4, TRPC6, RGS5. Marker genes for pulmonary ionocytes included CFTR, FOX11, ASCL3, ATP6V1G3, BSND, HEPACAM2. Marker genes for lung smooth muscle cells included PLN, DES, TNNT2, ACTG2, ATPIA2, MYH11, TAGLN, ACTA2, CALD1, NOTCH3. Marker genes for lung T and natural killer cells included CD4, CD3D, CD3E, CD3G, IL7R, CD8A, CD8B, FOXP3, TIGIT, CD27, NCAM1, KLRF1, GNLY, NKG7, and TNFRSF18.

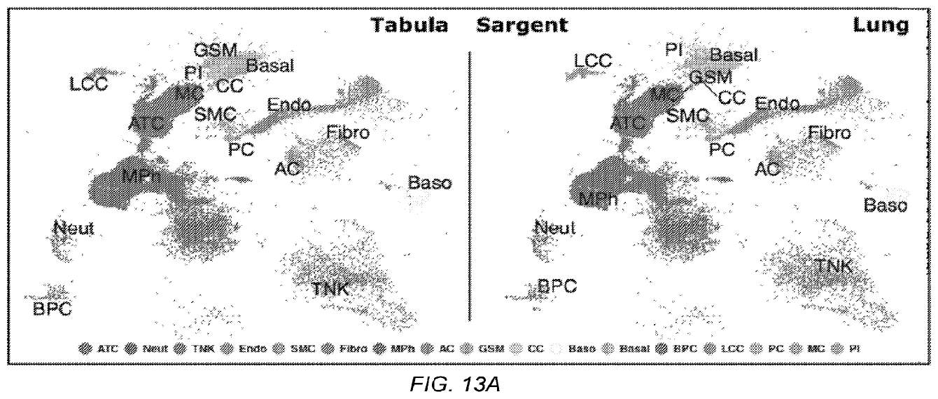

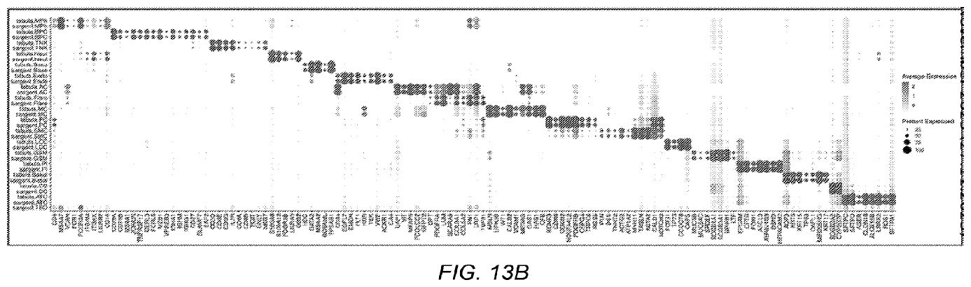

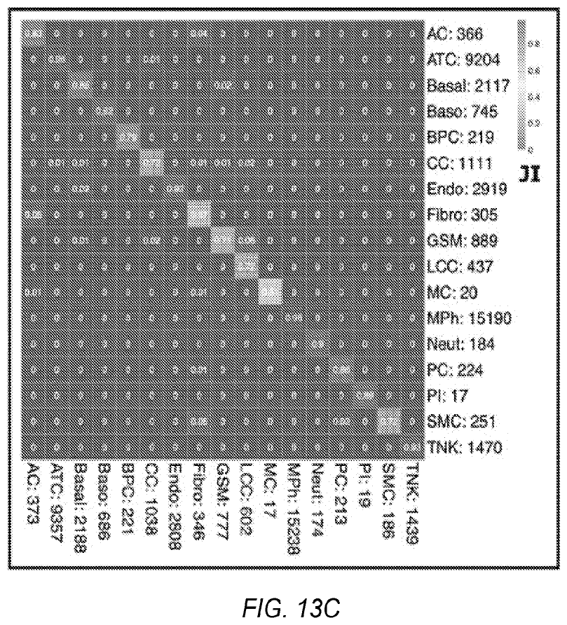

As shown in FIGS. 13A and 13B, the cell-type identification method was able to achieve a high similarity, 96%, with the manually annotated cells of the Tabula study over all cell types. Only 14 cells remained unlabeled with no strong indication among listed cell types (FIG. 13B). As shown in FIG. 13C, the high assignment similarity between the cell-type identification method and manual annotation was maintained at individual cell type level.

### Example 3: Computational Efficiency

Due to the large size of typical scRNA-seq datasets, increasing computational efficiency advantageous for cell-type identification methods. Processing time for the cell-type identification methods disclosed herein was evaluated using one single core with a 3.1 GHz processor and 128 GB Memory. Annotation of 15,000 simulated cells (the largest simulated data used in this study) by the cell-type identification methods disclosed herein took less than 2 minutes. In addition, the annotation of ~27 k±20 k (mean±standard deviation) experimental cells (the average data size used in this study) took ~2.3±1.7 min.

### Other Embodiments

It is to be understood that while the methods and compositions have been described in conjunction with the detailed description thereof, the foregoing description is intended to illustrate and not limit the scope of the methods and compositions, which is defined by the scope of the appended claims. Other aspects, advantages, and modifications are within the scope of the following claims.

What is claimed is:

1. A method of identifying a cell-type origin of each cell of a plurality of cells, the method comprising:
receiving a plurality of sequencing read counts;
providing a set of cell types to be detected and an associated gene set G for each cell type, such that each gene set G comprises a plurality of genes g;
scoring the sequencing read counts to generate an assignment score; and
assigning each cell of the plurality of cells to a cell-type with the highest assignment score to identify the cell-type origin of each cell.

2. The method of claim 1, further comprising trimming the sequencing read counts to remove ambiguous calls.

3. The method of any one of claims 1-2, further comprising smoothing annotations of the sequencing read counts.

4. The method of any one of claim 1-3, wherein the plurality of sequencing read counts are generated by RNA sequencing (RNA-seq).

5. The method of any one of claims 1-4, wherein the plurality of sequencing read counts are generated by single cell RNA sequencing (scRNA-seq) of each cell of the plurality of cells.

6. The method of any one of claims 1-5, wherein the scoring step comprises sorting the plurality of genes g of the associated gene set G exhibiting non-zero expression from high to low expression.

7. The method of any one of claims 1-6, wherein the scoring step further comprises generating, from the sorted genes of the associated gene set, a ranked vector of length N.

8. The method of claim 7, wherein the scoring step further comprises converting the ranked vector of length N to a binary sequence s, such that genes g that are included in a specific gene-set G are substituted by 1 or 0.

9. The method of claim 8, wherein the binary sequence s is represented by the following formula:
$$s = {\{ \begin{matrix}1 : {g}_{n} ∈ G \\ 0 : {g}_{n} ∉ G\end{matrix} \}}_{1 \leq n \leq N} .$$

10. The method of any one of claims 8-9, wherein the scoring step further comprises performing a cumulative sum over the binary sequence s up to each element k of the binary vector.

11. The method of claim 10, wherein the scoring step further comprises performing a sum over all generated sequential 1-to-k partial sums to produce an assignment-score S for a given cell according to the following formula:
$$S = {\sum }_{k = 1}^{N} {\sum }_{n = 1}^{k} {s}_{n} .$$

12. The method of any one of claims 8-11, wherein the scoring step further comprises performing the steps of claim 8-11 for each cell of the plurality of cells and gene sets for a total of M gene sets.

13. The method of any one of claims 1-12, wherein the scoring step further comprises transforming an input gene-by-cell expression matrix into a gene-set-by-cell assignment-score matrix, wherein the input gene-by-cell expression matrix comprises [number of genes×number of cells] and the gene-set-by-cell assignment-score matrix comprises [number of gene sets×number of cells].

14. The method of any one of claims 12-13, wherein each cells of the plurality of cells is assigned to a cell-type representing the highest assignment score represented by the following formula:
$$max {\{ {S}_{m} \}}_{1 \leq m \leq M} .$$

15. The method of any one of claims 1-14, wherein the trimming step further comprises calculating a Gini index value for each cell of the plurality of cells.

16. The method of any one of claims 1-14, wherein the trimming step further comprises transforming the gene-set-by-cell assignment-score matrix into a distribution of indexes according to the following formula:
∈[0,1]

17. The method of any one of claims 15-16, wherein the trimming step further comprises excluding cells with an index that is an outlier and/or is less than 0.5.

18. The method of any one of claims 1-17, wherein the smoothing step further comprises applying a k-nearest neighbor (kNN) algorithm to the sequencing read counts.

19. The method of claim 18, wherein the smoothing step further comprises:
identifying cells with a minimum of k nearest-neighbors; and
if more than 50% of neighbors reach a consensus annotation, annotating the neighboring cell according to the neighbor's consensus.

20. The method of any one of claims 1-19, further comprising providing a set of negative markers, wherein cells that do not express one or more negative markers are rewarded and cells that express one or more negative markers are penalized.

21. The method of any one of claims 1-20, wherein the method does not comprise normalization of the plurality of sequencing read counts.

22. The method of any one of claims 1-21, wherein the method does not comprise transformation of the plurality of sequencing read counts.

23. The method of any one of claims 1-22, wherein the method does not comprise clustering of the plurality of sequencing read counts.

24. The method of any one of claims 1-23, wherein the plurality of cells are human cells.

25. The method of any one of claims 1-24, wherein the set of cell types to be detected, comprises 1, 2, 3, 4, 5, 6, 7, 8, 9, or 10 cell types to be detected.

26. The method of any one of claims 1-25, wherein the plurality of genes g comprises 10, 20, 30, 40, 50, 60, 70, 80, 90, or 100 genes.

27. A computer program product tangibly embodied on a computer-readable medium, comprising instructions, that when executed by one or more processors, are configured to:
receive a plurality of sequencing read counts;
receive a set of cell types to be detected and an associated gene set G for each cell type, such that each gene set G comprises a plurality of genes g;
score the sequencing read counts to generate an assignment score; and
assign each cell of the plurality of cells to a cell-type with the highest assignment score to identify the tissue-type origin of each cell.

28. The method of claim 27, further comprising instructions that, when executed by the one or more processors, are configured to trim the sequencing read counts to remove ambiguous calls.

29. The method of any one of claims 27-28, further comprising instructions that, when executed by the one or more processors, are configured to smooth annotations of the sequencing read counts.
